(19) United States
(12) Patent Application Publication
(10) Pub. No.: US 2021/0301306 A1
(43) Pub. Date: Sep. 30, 2021

(54) GENE THERAPY METHODS TO CONTROL ORGAN FUNCTION

(71) Applicant: Cornell University, Ithaca, NY (US)

(72) Inventors: Michael G. Kaplitt, New York, NY (US); Roberta Marongiu, Long Island City, NY (US)

(21) Appl. No.: 17/264,752

(22) PCT Filed: Jul. 31, 2019
(86) PCT No.: PCT/US2019/044290

§ 371 Date: Jan. 29, 2021

Related U.S. Application Data
(60) Provisional application No. 62/712,669, filed on Jul. 31, 2018.

(51) Int. Cl. C12N 15/86; C07K 14/705; A61K 38/17; A61P 11/14; A61P 25/00; A61K 9/00

(52) U.S. Cl. CPC C12N 15/86; C07K 14/705; A61K 38/177; C12N 2750/14143; A61P 25/00; A61K 9/0019; A61K 9/0073; A61P 11/14

(57) ABSTRACT

Methods and compositions for controlling visceral organ function are provided. For example, the methods and compositions are useful to prevent, inhibit or treat disease as a result of controlling, e.g., regulating, organ function. In one embodiment, viral vectors are delivered to an organ, and the virus infects a nerve that regulates a function of the organ. In one embodiment, the vial vector is a retrograde vector. In one embodiment, the viral vector encodes a gene product, the activity of which is controlled by an exogenously delivered agent or energy. The delivery of the agent or energy thus controls the organ function.

### CROSS-REFERENCE TO RELATED APPLICATIONS

This application claims the benefit of the filing date of U.S. application No. 62/712,669, filed on Jul. 31, 2018, the disclosure of which is incorporated by reference herein.

### BACKGROUND

Gene therapy has shown great promise for a variety of neurological diseases. Recently it has become recognized that neuronal control of organ function represents an opportunity for therapeutic regulation of organ function through manipulation of neuronal activity. Mechanical approaches to altering neuronal function are currently available, including stimulators which can electrically stimulate either the main trunk or branches of nerves such as the vagus nerve. However, these non-specifically influence all neurons within the nerve being stimulated, and they involve complex implants that have complications inherent in mechanical devices, including lead migration and infection, while a pulse generator must also be regularly charged and/or periodically replaced in order to maintain function.

Gene therapy agents are capable of targeting neurons to visceral organs, yet injection of viral vectors into ganglia, brain or spinal cord regions harboring cell bodies for these neurons will not permit control of individual organs, since these are mostly mixed populations which send neurons to many organs. For example, sensory neurons of the vagus nerve from the stomach can sense stretch and satiety, while sensory neurons from the vagus to the lung are responsible for the cough reflex. Therefore, injection of gene therapy agents to modulate neuronal function into the nodose ganglion would target both populations of neurons, thereby influencing the function of both organs which would be undesirable when treating cough or a metabolic disorder alone.

Accordingly, there is a need for an approach to genetic modulation of the function of subsets of neurons to particular organs in order to regulate organ function to improve disease.

### SUMMARY

The disclosure provides materials and methods useful for control of organ function to prevent, inhibit or treat disease. In one aspect, the method provides for delivery of viral vectors to organs which are then taken up by the axons of nerves which regulate function of those organs. In one embodiment, the viral vector is an adeno-associated virus (AAV) vector. In one embodiment, a retrograde form of adeno-associated virus (AAV), when injected into the wall of the stomach, is specifically taken up into a subset of vagus nerve sensory neurons which respond to distention of the stomach and cause satiety. Molecules that provide for retrograde forms of vectors are known to the art and include but are not limited to native viral proteins, such as HSV protein, rabies virus G, glycoprotein type C, VSV G, B19G, pseudorabies virus protein, AAV capsid protein, and dynein. This represents a specific subset of neurons emanating from the nodose ganglion, while not affecting other nodose neurons which provide sensation to other visceral organs. The viral vector can also be other forms of retrograde vectors, including but not limited to retrograde lentiviral (LV) vectors, herpes simplex virus (HSV) vectors or canine adenovirus (CAV) vectors.

In one embodiment, a method is provided to deliver one or more genes to nerve fibers that control function of an organ, e.g., a visceral organ including but not limited to stomach, small intestine, large intestine, pancreas, liver, spleen, gall bladder, lung, kidney, and heart. In one embodiment, a viral gene therapy vector is delivered into a region of an organ that is innervated by a regulatory nerve such as a vagus nerve, cardiopulmonary nerve, thoracic splanchnic nerve, lumbar splanchnic nerve, sacral splanchnic nerve or pelvic splanchnic nerve. In one embodiment, the organ is a stomach, intestines, pancreas, liver, lung, heart, adrenal, kidney, gonads, bladder, anal sphincter or urinary sphincter. In one embodiment the viral vector is modified for retrograde transport in the central nervous system. In one embodiment, the viral vector is injected into the organ. In one embodiment, the delivery of the vector prevents, inhibits or treats a disease. In one embodiment, the viral vector is injected into the stomach and the expression of the gene controls food intake. In one embodiment, the viral vector is delivered to the lung. e.g., via inhalation, and the expression of the gene controls cough. In one embodiment, expression of the gene activates the regulatory nerve. In one embodiment, expression of the gene inhibits the regulatory nerve.

The disclosure also provides for a viral vector with improved properties for retrograde uptake into target neurons. This vector contains a capsid, which contains a mix of capsid proteins from one serotype of AAV, e.g., AAV serotype 2, with point mutations increasing retrograde uptake (retroAAV) (Tevro, et al., Neuron 92:372-378 (2016), which is incorporated by reference herein) and capsid proteins from a different serotype of AAV, e.g., AAV serotype rh10. This capsid mix creates a viral vector that has dramatically enhanced retrograde uptake and efficiency into afferent neurons compared with capsids that contain protein exclusively from retroAAV. This vector provides for efficient control of organ function.

The disclosure also provides a method for regulated control of organ function. The method generally includes delivery of a gene via a retrograde vector to neurons afferent to the organs, the product of which responds to an external drug or stimulus to control organ function. In one example, this method can be used to induce satiety and reduce food intake in order to control body weight. In this example, retrograde AAV (retroAAV) expressing a Designer Receptor Exclusively Activated by Designer Drugs (DREADD) that activates neurons, is injected into the wall of the stomach and is taken up into vagus sensory neurons, afferent to the stomach, which respond to stomach stretch and induce satiety. Following systemic administration of a DREADD activator. e.g., clozapine-N-oxide (CNO), satiety is induced and food intake is decreased. Other examples include delivery of an excitatory chemogenetic ion channel to these neurons followed by activation with the appropriate drug or delivery of the excitatory optogenetic ion channel ChR2 followed by light delivery to the nerve fibers to activate ChR2. In another example, this method is used to control intractable cough that is not due to an otherwise treatable disease. In one example, retrograde AAV expressing an inhibitory DREADD is aerosolized and inhaled for uptake into vagus sensory neurons of the lung, followed by systemic administration of a DREADD activator. e.g., CNO, to inhibit activity of these sensory neurons to reduce the cough reflex. In one example, retrograde AAV expressing, for instance, an inhibitory DREADD, is injected, e.g., into the vagus verve or nodose ganglion, followed by systemic administration of a DREADD activator, e.g., CNO, to inhibit activity of these sensory neurons to reduce the cough reflex.

In one embodiment, the viral vector encodes hM4Di, an engineered version of the M4 muscarinic acetylcholine receptor which, when bound by CNO, clozapine, perlapine, or compound 21 (see Chen et al., ACS Chem. Neurosic., 6:476 (2015)), which is incorporated by reference herein, results in membrane hyperpolarization through a decrease in cAMP signaling and increased activation of inward rectifying potassium channels. This yields a temporary suppression of neuronal activity similar to that seen after endogenous activation of the M4 receptor. hM3Dq (hD3q) is an engineered version of the M3 muscarinic receptor, which when activated by CNO, leads to activation of the phospholipase C cascade altering intracellular calcium and leading to burst-like firing of neurons. rM3Ds result in neuronal depolarization based on G-protein signaling (e.g., cAMP increases) which can modulate neuronal activity through arrestin-based signaling processes instead of G-protein signaling. Other options for neuronal excitation are other rM3Ds, which similarly result in neuronal depolarization based on G-protein signaling (e.g., cAMP increases) (see, e.g., Dong, Allen. Farrell. & Roth, 2010; Ferguson, Phillips, Roth, Wess, & Neumaier, 2013), and Rq(R165L), which can modulate neuronal activity through arrestin-based signaling processes instead of G-protein signaling. Another receptor is inhibitory DREADD receptor Pdi. A mutated form of the Gi-coupled kappa opioid receptor (KORD) is activated by salvinorin B (SalB) and so may also be employed in the viral vectors.

The disclosure also provides for a method of preventing spread of toxic proteins from the gastrointestinal tract to the brain through transfer of genes to the vagus nerve which prevent toxic protein transfer. This may occur through direct injection of viral vectors into sensory ganglia for the vagus nerve, such as the nodose ganglion, or direct injection of viral vectors into vagal efferent cell bodies in the brain, such as the dorsal motor nucleus of the vagus, or through injection of viral vectors into the wall of the gastrointenstinal tract or through oral administration, such vectors then being taken up into vagal nerve axons and transported retrograde to express a therapeutic agent within the cell bodies. In one example, a shRNA directed against alpha-synuclein, which prevents expression of alpha-synuclein protein in target neurons, is expressed from retrograde form of virus, e.g., a retroAAV/rh10 vector, delivered to vagal sensory fibers through injection into the wall of the stomach and/or intestines. The resulting expression of the shRNA within the sensory neurons of the vagus bk>cks expression of endogenous alpha-synuclein within these neurons, with the resulting prevention of spread of toxic synuclein pathology from pathological fibrils in the gastrointestinal tract which results in widespread brain pathology observed in Parkinson's disease, since expression within neurons is required for propagation of pathological synuclein. In another example, the retroAAV/rh10 vector within the vagal sensory fibers delivered through the gastrointestinal tract expresses an antibody directed against alpha-synuclein protein to prevent spread of alpha-synuclein. In one embodiment, the rAAV has a capsid having at least 80%, 85%, 90%, 92%, 95%, 96%, 97%, 98% or 99% amino acid sequence identity to SEQ ID NO:5 or 7.

### BRIEF DESCRIPTION OF THE DRAWINGS

FIG. 1 depicts expression of the fluorescent mCherry protein in afferent (sensory) fibers in the region of the dorsal motor nucleus of the vagus in the brain following injection of AAV vectors to the stomach wall. A known retrograde tracer (cholera toxin subunit B) was injected into the stomach to label afferent motor fibers. Injection of a mixed vector of AAV serotypes 2 and 1 (AAV2/1) showed virtually no neuronal uptake. Injection of a retroAAV, which shows increased retrograde uptake in the brain, resulted in mCherry protein expression within neuronal fibers of the dorsal motor nucleus, not cell bodies, suggesting that these are sensory fibers emanating from the nodose ganglion. The same procedure performed with a hybrid retroAAV and AAVrh10 capsid showed increased numbers of fibers expressing mCherry in the same brain region.

FIG. 2A depicts expressing of mCherry protein selectively within the nodose ganglion, which harbors cell bodies for vagal sensory neurons, following injection of AAV vectors into the stomach wall. The cholera toxin tracer shows labeling of a small number of neuronal cell bodies within the nodose ganglion. Virtually no positive cell bodies were observed with AAV2/1, even though we have observed some retrograde uptake into neurons in the brain. retroAAV shows an increased number of cell bodies within the nodose ganglion expressing mCherry, indicating effective retrograde uptake, yet the uptake is selective as only a subset of nodose neurons are labelled. The retroAAV/rh10 hybrid vector demonstrates more labelling of neuronal cell bodies in the nodose compared with retroAAV alone, even though these still represent a subset of neurons FIG. 2B. Quantification of neuronal cell counts shows a roughly 3 fold increase in the number of positive neurons within the nodose ganglion following retroAAV/rh10 administration into the stomach wall compared with retroAAV alone.

FIG. 3 depicts the response of normally fed fasted mice injected into the stomach wall with retroAAV/rh10 expressing a DREADD (hD3q) which activates neurons in response to 1 mg/kg CNO. Animals injected with retroAAV/rh10 expressing the marker gene mCherry fed normally in response to CNO. Animals injected with retroAAV/rh10 expressing the DREADD but given saline instead of CNO had identical normal feeding for 6 and 24 hrs after saline administration compared with the mCherry animals given CNO. Animals injected with retroAAV/rh10 expressing the DREADD and then given CNO showed significantly reduced food intake for several hours after CNO administration. When the CNO was cleared, feeding behavior returned to normal, with total food intake over 24 hours being reduced compared with control due to a reduction in food intake for 4-6 hours after CNO, while the period after CNO cleared from 6-24 hours showed similar food intake to controls over that period.

FIGS. 4A-4B the response of mice injected into the stomach wall with retroAAV/rh10 expressing a DREADD (hD3q) and starved for 24 hours prior to administration of either CNO or saline. Animals injected with retroAAV/rh10 expressing the marker gene mCherry had increased feeding compared with normally fed mice (compare with FIG. 3) for 6 hrs and 24 hrs after administration of CNO. Animals injected with retroAAV/rh10 expressing the DREADD but given saline instead of CNO had identical feeding for 6 and 24 hrs after saline administration compared with the mCherry animals given CNO. Animals injected with retroAAV/rh10 expressing the DREADD and then given CNO showed significantly reduced food intake for several hours after CNO administration despite animals having been starved for the preceding 24 hrs. The effect was so profound that intake of food for 4-6 hrs after CNO administration in this starved group was still lower than animals which had been normally fed prior to testing and then received control vectors or drug (see FIG. 3). When the CNO was cleared, feeding behavior rebounded in this group compared with the normally fed group, with animals having increased food intake from 6-24 hrs after CNO had cleared, due to the period of starvation that was effectively extended by the treatment compared with controls. This was different than normally fed mice, where normal food intake resumed after the CNO was cleared, but there was no increased intake compared with controls.

FIG. 5 illustrates an exemplary approach to deliver gene therapy to control organ function.

FIG. 6 depicts data on feeding behavior in fasted mice with 3 mg/kg CNO.

FIG. 7 shows data for feeding behavior in normally fed mice with 3 mg/kg CNO.

FIGS. 8A-8E depict feeding behavior data in normally fed mice with 1 mg/kg CNO.

FIGS. 9A-9E show long term stability of reduced feeding behavior in normally fed mice with 1 mg/kg CNO.

FIG. 10 shows the reduced weight gain in gut retro/rh10AAV HD3q (DREADD) mice on 60% high fat diet treated with daily 1 mg/kg CNO (C) compared with saline (S).

FIG. 11 depicts the continued reduced weight gain in gut retro/rh10AAV HD3q (DREADD) mice on 60% high fat diet treated with daily 1 mg/kg CNO (C) compared with saline (S). X-axis shows chronological days following initiation of drug therapy, day 33 is day 24 in previous figures.

FIGS. 12A-12M illustrate the sequence for pNLRep2_RETRO Cap2 (SEQ ID NO:1).

FIGS. 13A-14B provides the sequence for pNLRep2_rh10Cap (SEQ ID NO:2).

FIGS. 14A-14B show the sequence for AAV.CBA.flag-mCheryy.WPRE (SEQ ID NO:3).

FIGS. 15A-15P provide exemplary sequences for the capsid of AAVrh0 (SEQ ID Nos. 4-5) and retroAAV2 (SEQ ID Nos. 6-7).

FIGS. 16A-16B provide sequences for human M3 and M4. DREADDs for hM3 may have a substitution at residue 149 and/or 239, e.g., Y149C or A239G in mM3, and DREADDS for hM4 may have substitutions at residue 113 and/or 203, e.g., Y113C or A203G in mM4 (SEQ ID NO:12).

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide. e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays. e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are increasingly preferred. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells.” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant.” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

### Gene Transfer Vectors

The disclosure provides a gene transfer vector, e.g., a viral gene transfer vector, useful to deliver genes to neurons or nerve fibers, or the spread of a gene product that is toxic, such as toxic protein, from the gastrointestinal tract to the brain. Various aspects of the gene transfer vector and method are discussed below. Accordingly, any combination of parameters can be used according to the gene transfer vector and the method.

A “gene transfer vector” is any molecule or composition that has the ability to carry a heterologous nucleic acid sequence into a suitable host cell where synthesis of the encoded protein takes place. Typically, a gene transfer vector is a nucleic acid molecule that has been engineered, using recombinant DNA techniques that are known in the art, to incorporate the heterologous nucleic acid sequence. Desirably, the gene transfer vector is comprised of DNA. Examples of suitable DNA-based gene transfer vectors include plasmids and viral vectors. However, gene transfer vectors that are not based on nucleic acids, such as liposomes, are also known and used in the art. The inventive gene transfer vector can be based on a single type of nucleic acid (e.g., a plasmid) or non-nucleic acid molecule (e.g., a lipid or a polymer). The gene transfer vector can be integrated into the host cell genome, or can be present in the host cell in the form of an episome.

In one embodiment, the gene transfer vector is a viral vector. Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York. N.Y. (1994).

In an embodiment, the invention provides an adeno-associated virus (AAV) vector. The AAV vector may include a gene to be expressed and additional components that do not materially affect the AAV vector (e.g., genetic elements such as poly(A) sequences or restriction enzyme sites that facilitate manipulation of the vector in vitro). Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell. 61:447 (1990)). These proteins also regulate transcription from endogenous AAV promoters and promoters within helper viruses (see, e.g., Pereira et al., J. Virol., 71:1079 (1997)). The other Rep proteins modify the function of Rep78 and Rep68. The cap genes encode the capsid proteins VP1, VP2, and VP3. The cap genes are transcribed from the p40 promoter.

The AAV vector may be generated using any AAV serotype known in the art. Several AAV serotypes and over 100 AAV variants have been isolated from adenovirus stocks or from human or nonhuman primate tissues (reviewed in, e.g., Wu et al., Molecular Therapy. 14(3): 316 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-6 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter. Hum. Gene Ther., 16:541 (2005); and Wu et al., supra). Genome sequences of various AAV serotypes and comparisons thereof are disclosed in, for example. GenBank Accession numbers U89790, J01901, AF043303, and AF085716; Chiorini et al., J. Virol., 21:6823 (1997); Srivastava et al., J. Virol., 45:555 (1983); Chiorini et al., J. Virol., 73:1309 (1999); Rutledge et al., J. Virol., 72:309 (1998); and Wu et al., J. Virol., 74:8635 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J. Virol., 23(2):939 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (e.g., functionally substitute) corresponding sequences from other serotypes during production of AAV particles in mammalian cells.

Generally, the cap proteins, which determine the cellular tropism of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 13(1):1 (2006); Gao et al., J. Virol., 78:6381 (2004); Gao et al., Proc. Natl. Acad. Sci. USA, 99:11854 (2002); De et al., Mol. Ther., 13:67 (2006); and Gao et al., Mol. Ther., 1.3:77 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). In one embodiment, the AAV vector is generated using an AAV that infects a non-human primate pseudotyped with an AAV that infects humans. Examples of such pseudotyped AAV vectors are disclosed in, e.g., Cearley et al., Molecular Therapy. 13:528 (2006). In one embodiment, an AAV vector can be generated which comprises a capsid protein from an AAV that infects rhesus macaques pseudotyped with AAV2 inverted terminal repeats (ITRs). In a particular embodiment, the inventive AAV vector comprises a capsid protein from AAV10 (also referred to as “AAVrh.10”), which infects rhesus macaques pseudotyped with AAV2 ITRs (see, e.g., Watanabe et al., Gene Ther., 17(8):1042 (2010); and Mao et al., Hum. Gene Therapy, 22:1525 (2011)).

In addition to the gene to be expressed, the AAV vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185. Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346 (1996)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 2:4324 (1999); Nuc. Acid. Res., 28:e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In one embodiment, the nucleic acid sequence is operably linked to a CMV enhancer/chicken beta-actin promoter (also referred to as a “CAG promoter”) (see, e.g., Niwa et al., Gene, 108:193 (1991); Daly et al., Proc. Natl. Acad. Sci. U.S.A., 9:2296 (1999); and Sondhi et al., Mol. Ther., 15:481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh.10, 8 or 9 as required). After 72 hours, the cells are harvested and the vector is released from the cells by five freeze/thaw cycles. Subsequent centrifugation and benzonase treatment removes cellular debris and unencapsidated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Pharmaceutical Compositions and Delivery

The invention provides a composition comprising, consisting essentially of, or consisting of the above-described gene transfer vector and a pharmaceutically acceptable (e.g., physiologically acceptable) carrier. When the composition consists essentially of the inventive gene transfer vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the inventive gene transfer vector and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of the gene transfer vector described herein. The composition can be frozen or lyophilized for storage and reconstituted in a suitable sterile carrier prior to use. The compositions can be generated in accordance with conventional techniques described in, e.g., Remington: The Science and Practice of Pharmacy. 21st Edition. Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80. L-arginine, polyvinylpyrrolidone, trehalose, and combinations thereof. Use of such a composition will extend the shelf life of the gene transfer vector, facilitate administration, and increase the efficiency of the inventive method. Formulations for gene transfer vector-containing compositions are further described in, for example. Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the inventive gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA, can be administered to enhance or modify the immune response. Antibiotics, i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene transfer vectors may be intracerebral (including but not limited to intraparenchymal, intraventricular, or intracisternal), intrathecal (including but not limited to lumbar or cisterna magna), or systemic, including but not limited to intravenous, or any combination thereof, using devices known in the art. Delivery may also be via surgical implantation of an implanted device.

The dose of the gene transfer vector in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as pathology, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. The therapeutically effective amount may be between 1×10¹⁰ genome copies to 1×10¹³ genome copies.

In one embodiment, the vector is an adenovirus, adeno-associated virus (AAV), retrovirus or lentivirus vector. In one embodiment, the AAV vector is pseudotyped. In one embodiment, the AAV vector is pseudotyped with AAVrh.10. AAV8, AAV9, AAV5, AAVhu.37, AAVhu.20, AAVhu.43, AAVhu.8, AAVhu.2, or AAV7 capsid. In one embodiment, the AAV vector is pseudotyped with AAVrh.10. AAV8, or AAV5. In one embodiment, the AAV vector is AAV2, AAV5. AAV7, AAV8, AAV9 or AAVrh.10. Further provided is a pharmaceutical composition comprising an amount of the gene therapy vector described above. A dose of the viral vector may be about 1×10¹¹ to about 1×10¹⁶ genome copies, about 1×10¹² to about 1×10¹⁵ genome copies about 1×10¹¹ to about 1×10¹³ genome copies, or about 1×10¹³ to about 1×10¹⁵ genome copies.

In one embodiment of the invention, the composition is administered once to the mammal. It is believed that a single administration of the composition will result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

### EXEMPLARY EMBODIMENTS

The disclosure provides materials and methods useful for control of organ function to prevent, inhibit or treat disease. In one aspect, the method provides for delivery of viral vectors to organs which are then taken up by the axons of nerves which regulate function of those organs. In one embodiment, the viral vector is a adeno-associated virus (AAV) vector. In one embodiment, a retrograde form of adeno-associated virus (AAV), when injected into the wall of the stomach, is specifically taken up into a subset of vagus nerve sensory neurons which respond to distention of the stomach and cause satiety. This represents a specific subset of neurons emanating from the nodose ganglion, while not affecting other nodose neurons which provide sensation to other visceral organs. The viral vector can also be other forms of retrograde vectors, including but not limited to retrograde lentiviral (LV) vectors, herpes simplex virus (HSV) vectors orcanine adenovirus (CAV) vectors.

The disclosure also provides for a viral vector with improved properties for retrograde uptake into target neurons. This vector contains a capsid, which contains a mix of capsid proteins from one serotype of AAV, e.g., AAV serotype 2, with point mutations increasing retrograde uptake (retroAAV) (Tevro, et al., Neuron 92:372-378 (2016), which is incorporated by reference herein) and capsid proteins from a different serotype of AAV, e.g., AAV serotype rh10. This capsid mix creates a viral vector that has dramatically enhanced retrograde uptake and efficiency into afferent neurons compared with capsids that contain protein exclusively from retroAAV. This vector provides for efficient control of organ function.

The disclosure also provides a method for regulated control of organ function. The method generally includes delivery of a gene via a retrograde vector to neurons afferent to the organs, the product of which responds to an external drug or stimulus to control organ function. In one example, this method can be used to induce satiety and reduce food intake in order to control body weight. In this example, retrograde AAV (retroAAV) expressing a Designer Receptor Exclusively Activated by Designer Drugs (DREADD) that activates neurons, is injected into the wall of the stomach and is taken up into vagus sensory neurons, afferent to the stomach, which respond to stomach stretch and induce satiety. Following systemic administration of a DREADD activator, e.g., clozapine-N-oxide (CNO), satiety is induced and food intake is decreased. Other examples include delivery of an excitatory chemogenetic ion channel to these neurons followed by activation with the appropriate drug or delivery of the excitatory optogenetic ion channel ChR2 followed by light delivery to the nerve fibers to activate ChR2. In another example, this method is used to control intractable cough that is not due to an otherwise treatable disease. In this example, retrograde AAV expressing an inhibitory DREADD is aerosolized and inhaled for uptake into vagus sensory neurons of the lung, followed by systemic administration of a DREADD activator, e.g., CNO, to inhibit activity of these sensory neurons to reduce the cough reflex.

The disclosure also provides for a method of preventing spread of toxic proteins from the gastrointestinal tract to the brain through transfer of genes to the vagus nerve which prevent toxic protein transfer. This may occur through direct injection of viral vectors into sensory ganglia for the vagus nerve, such as the nodose ganglion, or direct injection of viral vectors into vagal efferent cell bodies in the brain, such as the dorsal motor nucleus of the vagus, or through injection of viral vectors into the wall of the gastrointenstinal tract or through oral administration, such vectors then being taken up into vagal nerve axons and transported retrograde to express a therapeutic agent within the cell bodies. In one example, a shRNA directed against alpha-synuclein, which prevents expression of alpha-synuclein protein in target neurons, is expressed from retrograde form of virus. e.g., a retroAAV/rh10 vector, delivered to vagal sensory fibers through injection into the wall of the stomach and/or intestines. The resulting expression of the shRNA within the sensory neurons of the vagus blocks expression of endogenous alpha-synuclein within these neurons, with the resulting prevention of spread of toxic synuclein pathology from pathological fibrils in the gastrointestinal tract which results in widespread brain pathology observed in Parkinson's disease, since expression within neurons is required for propagation of pathological synuclein. In another example, the retroAAV/rh10 vector within the vagal sensory fibers delivered through the gastrointestinal tract expresses an antibody directed against alpha-synuclein protein to prevent spread.

In one embodiment, a method of delivering genes to nerve fibers controlling organ function is provided, which method comprises delivering a gene therapy vector into regions of target organs innervated by the regulatory nerves. In one embodiment, the nerve fibers are selected from a group consisting of the vagus nerve, cardiopulmonary nerves, thoracic splanchnic nerves, lumbar splanchnic nerves, sacral splanchnic nerves and pelvic splanchnic nerves. In one embodiment, the organ is selected from a group consisting of stomach, intestines, pancreas, liver, lung, heart, adrenal, kidney, gonads, bladder, anal and urinary sphincters. In one embodiment, the gene therapy vector is a viral vector selected from a group consisting of adeno-associated virus, lentivirus, adenovirus, herpes simplex virus. In one embodiment, the gene therapy vector is a modified viral vector selected for retrograde transport in the central nervous system, including retrograde forms of adeno-associated virus and lentivirus and canine adenovirus.

In one embodiment, a method of regulating the function of an organ to improve disease, which method comprises delivering a gene capable of modulating neuronal activity to the nerve controlling the organ through injection of a gene therapy vector into the organ for uptake and retrograde transport in the neuron. In one embodiment, the nerve fiber is selected from a group consisting of the vagus nerve, cardiopulmonary nerves, thoracic splanchnic nerves, lumbar splanchnic nerves, sacral splanchnic nerves and pelvic splanchnic nerves. In one embodiment, the organ is selected from a group consisting of stomach, intestines, pancreas, liver, lung, heart, adrenal, kidney, gonads, bladder, anal and urinary sphincters. In one embodiment, the viral vectors selected from a group consisting of adeno-associated virus, lentivirus, adenovirus, herpes simplex virus. In one embodiment, modified viral vectors selected for retrograde transport in the central nervous system, including retrograde forms of adeno-associated virus and lentivirus and canine adenovirus, are employed. In one embodiment, the gene being delivered regulates the activity of the nerve controlling the organ in response to an exogenous agent or stimulus. In one embodiment, the gene being delivered encodes one or more light-sensitive ion channels (optogenetics), chemically-responsive ion channels (chemogenetics), ultrasound-sensitive ion channels (sonogenetics), magnetic-field responsive ion channels (magnetogenetics) and designer receptors exclusively activated by designer drugs (DREADDs).

In one embodiment, a method of controlling food intake is provided by delivering a viral vector to vagus nerve fibers by injection of vectors into visceral organs to reduce food intake. In one embodiment, the organ is the stomach, duodenum or small intestine. In one embodiment, the gene being delivered regulates the activity of the nerve controlling the organ in response to an exogenous agent or stimulus. In one embodiment, the gene being delivered is s encodes oneor more of light-sensitive ion channels (optogenetics), chemically-responsive ion channels (chemogenetics), ultrasound-sensitive ion channels (sonogenetics), magnetic-field responsive ion channels (magnetogenetics) and designer receptors exclusively activated by designer drugs (DREADDs).

In one embodiment, a method of preventing cough is provided by delivering a viral vector to nerve fibers of the lung via inhalation of viral vectors. In one embodiment, the gene being delivered regulates the activity of the nerve controlling the organ in response to an exogenous agent or stimulus. In one embodiment, the gene encodes one or more proteins including one or more of light-sensitive ion channels (optogenetics), chemically-responsive ion channels (chemogenetics), ultrasound-sensitive ion channels (sonogenetics), magnetic-field responsive ion channels (magnetogenetics) and designer receptors exclusively activated by designer drugs (DREADDs).

In one embodiment, a viral vector for improved retrograde delivery and uptake into neurons controlling visceral organs is provided. In one embodiment, the vector comprises an adeno-associated viral vector with a capsid comprising a mix of capsids, e.g., from AAV retro and AAVrh10.

In one embodiment, a method of preventing spread of toxic proteins from the gastrointestinal tract to the brain through delivery of viral vectors to the vagus nerve expressing genes capable of blocking toxic protein spread is provided. In one embodiment, the viral vector is taken up retrograde from the gastrointestinal system. In one embodiment, the toxic protein being targeted is selected from a group consisting of alpha-synuclein, tau, beta-amyloid, or huntingtin. In one embodiment, the gene being expressed from the vagus nerve to prevent toxic protein spread is selected from a group consisting of small hairpin RNA (shRNA), microRNA (miRNA), CrispR/Cas9, antibodies, single-chain antibodies, or intrabodies.

In one embodiment, a method of preventing, inhibiting or treating a cough in a mammal is provided. The method includes delivering, e.g., injecting, a composition comprising a viral vector comprising a gene encoding a protein, the activity of which is inhibited by administration of an exogenous agent or delivery of energy, indirectly to parasympathetic nerve fibers that innervate the lung of the mammal; and exposing the mammal to the agent or energy in an amount effective to prevent, inhibit or treat a cough in the mammal. In one embodiment, the composition is administered via inhalation. In one embodiment, the mammal is a human. In one embodiment, the mammal has idiopathic cough or intractable cough. In one embodiment, the mammal has chronic obstructive pulmonary disease (COPD) or gastric reflux. In one embodiment, the viral vector is an adeno-associated virus, lentivirus, adenovirus, or herpes simplex virus vector. In one embodiment, the virus is a retrograde form of adeno-associated virus (AAV), lentivirus or canine adenovirus. In one embodiment, the virus is modified for retrograde transport. In one embodiment, the AAV has a capsid comprising proteins from more than one serotype of AAV. In one embodiment, the capsid proteins are AAV2 and AAVrh10. In one embodiment, the capsid proteins are AAV5 and AAVrh10. In one embodiment, the capsid proteins are AAV2 and AAV5. In one embodiment, the gene encodes a light-sensitive ion channel (optogenetics), a chemically-responsive ion channel (chemogenetics), an ultrasound-sensitive ion channel (sonogenetics), a magnetic-field responsive ion channel (magnetogenetics) or a designer receptor exclusively activated by designer drugs (DREADDs).

In one embodiment, a method of preventing, inhibiting or treating a cough is provided comprising delivering a composition comprising an effective amount of a composition comprising a viral vector comprising a gene, to nerve fibers of a mammalian lung. In one embodiment, the expression of the gene inhibits neuronal activity. In one embodiment, the gene encodes a DREADD or some other chemogenetic channel, GAD for production of GABA to inhibit a neuron, or a siRNA to block an excitatory protein or channel. In one embodiment, the composition is administered via inhalation or injection. e.g., directly into the vagus nerve or the nodose ganglion. In one embodiment, the gene regulates the activity of the nerve controlling the organ in response to an exogenous agent or stimulus. In one embodiment, the mammal is a human. In one embodiment, the mammal has idiopathic cough or intractable cough. In one embodiment, the mammal has COPD or gastric reflux. In one embodiment, the viral vector is an adeno-associated virus, lentivirus, adenovirus, or herpes simplex virus vector. In one embodiment, the virus is a retrograde form of adeno-associated virus, lentivirus or canine adenovirus. In one embodiment, the virus is modified for retrograde transport. In one embodiment, the gene encodes a light-sensitive ion channel (optogenetics), a chemically-responsive ion channel (chemogenetics), an ultrasound-sensitive ion channel (sonogenetics), a magnetic-field responsive ion channel (magnetogenetics) or a designer receptor exclusively activated by designer drugs (DREADDs). In one embodiment, the viral vector is a rAAV comprising a chimeric adeno-associated viral capsid comprising two or more different AAV capsid serotypes. In one embodiment, the vital vector transduces vagal afferents. In one embodiment, one of the AAV serotypes comprises AAV2. In one embodiment, one of the AAV serotypes comprises AAVrh10.

In one embodiment, a method of delivering genes to nerve fibers to control visceral organ function in a mammal is provided. In one embodiment, a visceral organ does not include brain or muscle. The method includes delivering a composition comprising a viral vector comprising a gene encoding a protein, the activity of which is inhibited by administration of an exogenous agent or delivery of energy, to one or more regions of a mammalian organ innervated by a regulatory nerve; and exposing the mammal to the agent or the energy in an effective amount. In one embodiment, the nerve fiber is the vagus nerve, cardiopulmonary nerve, thoracic splanchnic nerve, lumbar splanchnic nerve, sacral splanchnic nerve or pelvic splanchnic nerve. In one embodiment, the mammalian organ to be controlled is a stomach, intestine, pancreas, liver, lung, heart, adrenal, kidney, gonad, bladder, anal sphincter or urinary sphincter. In one embodiment, the composition is administered to is a stomach, intestine, pancreas, liver, lung, heart, adrenal, kidney, gonad, bladder, anal sphincter or urinary sphincter, or to a blood vessel, duct or other cavity. In one embodiment, the viral vector is an adeno-associated virus, lentivirus, adenovirus, or herpes simplex virus vector. In one embodiment, the viral vector provides for retrograde transport in neurons. In one embodiment, the virus is modified to provide for retrograde transport in the central nervous system. In one embodiment, the virus is a retrograde form of adeno-associated virus, lentivirus or canine adenovirus. In one embodiment, the gene regulates the activity of the nerve controlling the organ in response to an exogenous agent or stimulus. In one embodiment, the gene encodes a light-sensitive ion channel (optogenetics), a chemically-responsive ion channel (chemogenetics), an ultrasound-sensitive ion channel (sonogenetics), a magnetic-field responsive ion channel (magnetogenetics) or a designer receptor exclusively activated by designer drugs (DREADDs). In one embodiment, the gene encodes a channelrhodopsin, e.g., TREK-1, nicotinic acetylcholine receptor, gramicidin A, a voltage-gated potassium channel, an ionotropic glutamate channel. Naᵥ1.5, KCNQ1, KCNA, MEC-4, a DEG/ENaC/ASIC ion channel, or a mechanosensitive ion channel (MscL) such as TRPV4. In one embodiment, the composition is delivered to the vagus nerve. In one embodiment, the amount administered allows for control of food intake in the mammal. In one embodiment, the visceral organ is a stomach, duodenum or small intestine. In one embodiment, the gene encodes a gene product capable of blocking toxic protein spread to the vagus nerve of the mammal. In one embodiment, the viral vector is taken up retrograde from the gastrointestinal system. In one embodiment, the toxic protein comprises alpha-synuclein, tau, beta-amyloid, or huntingtin. In one embodiment, the gene encodes small hairpin RNA (shRNA), microRNA (miRNA). CrispR/Cas9, an antibody, a single-chain antibody, or an intrabody. In one embodiment, the amount administered prevents or inhibits spread of a toxic protein from the gastrointestinal tract to the brain in a mammal. In one embodiment, the mammal is a human. In one embodiment, the viral vector is an adeno-associated virus, lentivirus, adenovirus, or herpes simplex virus vector. In one embodiment, the virus is modified to provide for retrograde transport in the central nervous system. In one embodiment, the virus is a retrograde form of adeno-associated virus, lentivirus or canine adenovirus. In one embodiment, one of the AAV serotypes comprises AAV2. In one embodiment, one of the AAV serotypes comprises AAVrh10. In one embodiment, the viral vector is a rAAV comprising a chimeric adeno-associated viral capsid comprising two or more different AAV capsid serotypes. In one embodiment, the amount administered prevents, inhibits or treats disease in a visceral organ in the mammal.

In one embodiment, a rAAV comprising a capsid formed of capsid proteins from two or more different AAV serotypes (a chimeric AAV capsid) is provided. In one embodiment, one of the serotypes comprises AAV2. In one embodiment, one of the serotypes comprises AAVrh10. In one embodiment, one of the capsid serotypes comprises AAV2 and another comprises AAVrh10. In one embodiment, one of the capsid serotypes comprises AAV2 and another comprises AAV1, AAV3, AAV5, AAV8 or AAV9. In one embodiment, one of the capsid serotypes comprises AAVrh0 and another comprises AAV1, AAV2, AAV3. AAV5, AAV8 or AAV9. In one embodiment, one of the capsid serotypes comprises AAV5 and another comprises AAV1, AAV2, AAV3, AAV8 or AAV9. In one embodiment, one of the capsid serotypes comprises AAV9 and another comprises AAV1, AAV2, AAV3, AAV5, or AAV8. In one embodiment, one of the capsid serotypes provides for retrograde delivery. In one embodiment, the rAAV encodes a therapeutic gene product, a prophylactic gene product or an exogenously activatable protein.

The invention will be further described by the following non-limiting examples.

### Example 1

The figures show a method to control feeding behavior via regulated gene therapy. Obesity is among most common public health problems. Over 700 million obese worldwide (BMI>30 kg/m²) prevalence has doubled in last 25 years (GBD obesity collaborators. Health Effects of Overweight and Obesity in 195 Countries Over 25 years, NEJM 377:13, 2017). Over 78 million obese in U.S. BMI>30 is associated with reduced longevity and increased risk for numerous diseases including diabetes, cardiovascular disease and cancer. Severe obesity (>40 kg/m²) represent a rapidly growing population with strong unmet need. Roughly million U.S. in 2010, projected to increase to 25 million US by 2025 (Finkelstein, et al Obesity and Severe Obesity Forecasts through 2030, Am J Prev Med 42:563, 2012). 5-15% loss of excess body weight reduces risk factors for cardiovascular and other diseases in randomized trials (Office of Surgeon General, Call to Action to Prevent and Decrease Overweight and Obesity, 2001).

Bariatric Surgery is major current option for patients with severe obesity. Most popular procedure is sleeve gastrectomy to reduce stomach size and induce early satiety. 228,000 bariatric surgeries performed in U.S. in 2017 despite 15-25 million people meeting criteria for surgery (BMI>40 or BMI 30-40 with serious weight-related health problems) (https://asmbs.org/resources/estimate-of-bariatric-surgery-numbers). Over 14% were revision surgeries. Post-surgical regimen requires 1-3 days in the hospital, then liquids only for 7 days, pureed foods for 3 weeks before return to regular diet (httpsJ/www.mavgclinic.org/tests-procedures/sleeve-gastrectomy/about/pac-20385183). Ongoing daily multivitamin and calcium supplement and monthly B12 injection required for life due to malabsorption. Bariatric surgery associated with 13-21% complication rate. Minimally invasive gene therapy approach would provide an outpatient procedure option with no need for post-procedure management and minimal risk of surgical complications.

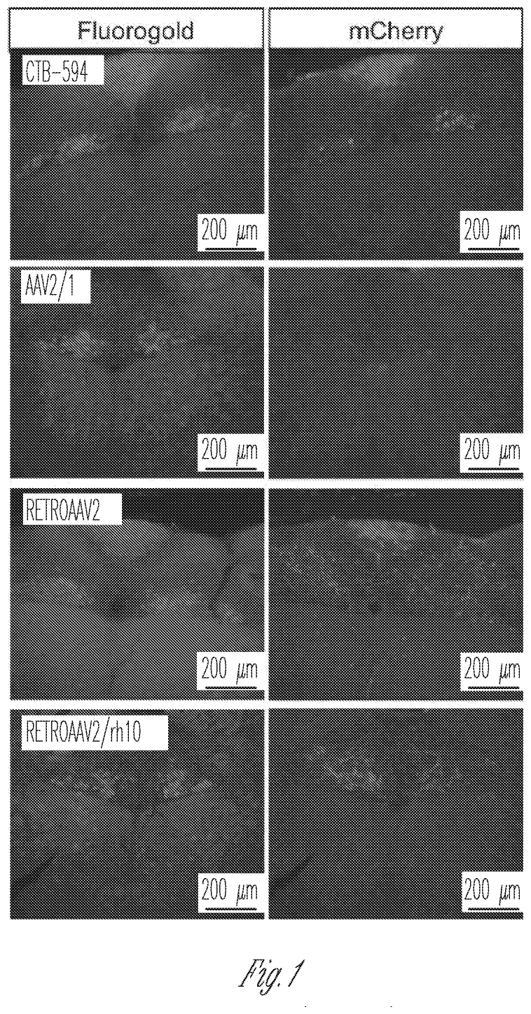

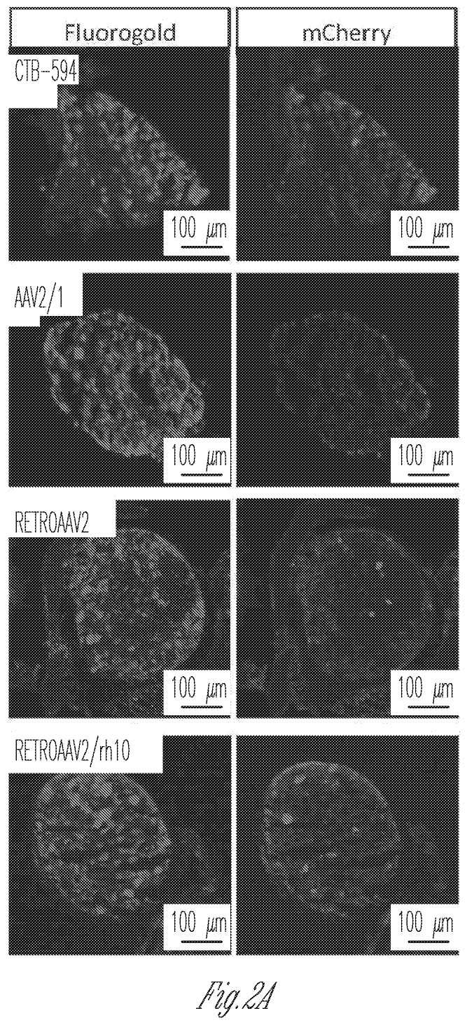

FIG. 1 shows data for AAV delivery to the gastrointestinal tract to target the dorsal nucleus of vagus nerve: mCherry signal in the dorsal nucleus of vagus nerve. Different AAV vectors were injected into the corpus of the stomach (1×10¹² p/ml) as well as fluorescent labeled cholera toxin subunit B (CTB-594) as a positive control. On the day of surgery, the mice also received an i.p. injection of retro-tracer Fluorogold to label the soma of afferent and efferent neurons in the gastrointestinal tract. Four weeks post-surgery nodose ganglia and brain were harvested for histological analysis of mCherry red fluorescent protein, which is expressed as a cassette under the chicken beta-actin promoter by the AAV vectors (AAV.CBA.mCherry).

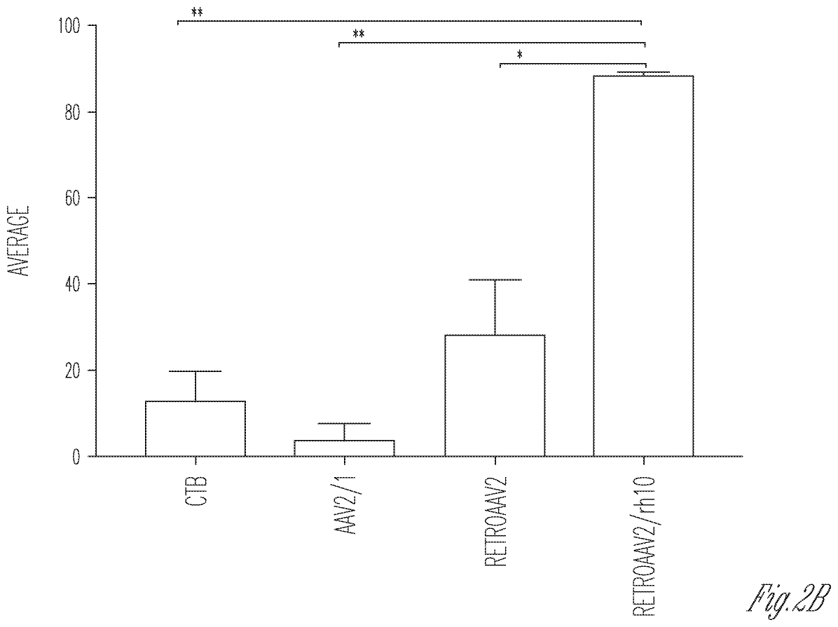

FIG. 2 illustrates AAV delivery to the gastrointestinal tract to target the dorsal nucleus of vagus nerve: mCherry signal in the nodose ganglion. Different AAV vectors were injected into the corpus of the stomach (1×10¹² p/ml) as well as fluorescent labeled cholera toxin subunit B (CTB-594) as a positive control. On the day of surgery, the mice also received an i.p. injection of retro-tracer Fluorogold to label the soma of afferent and efferent neurons in the gastrointestinal tract. Four weeks post-surgery nodose ganglia and brain were harvested for histological analysis of mCherry red fluorescent protein, which is expressed as a cassette under the chicken beta-actin promoter by the AAV vectors (AAV.CBA.mCherry).

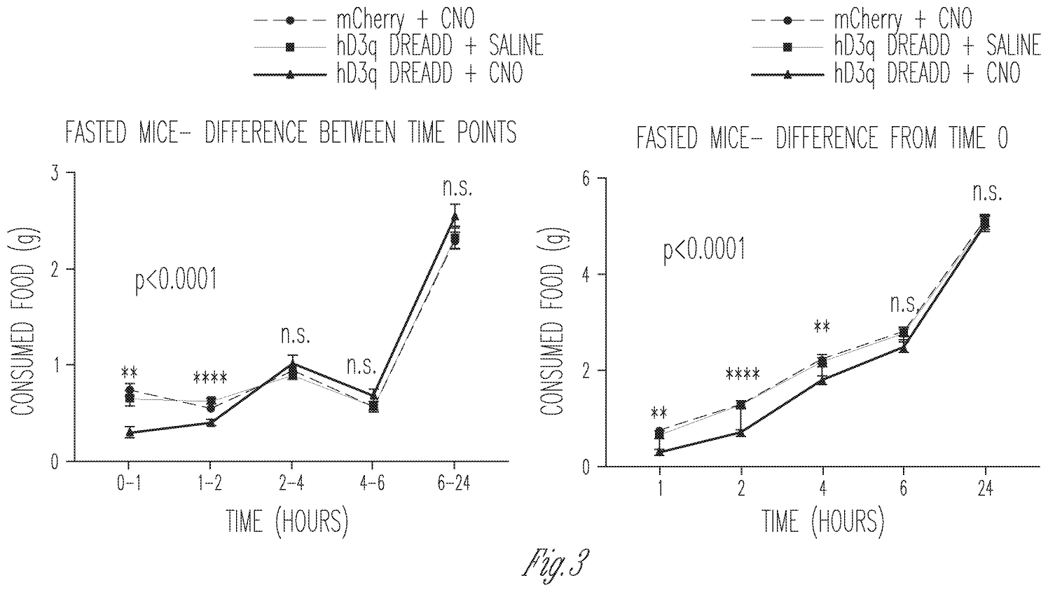

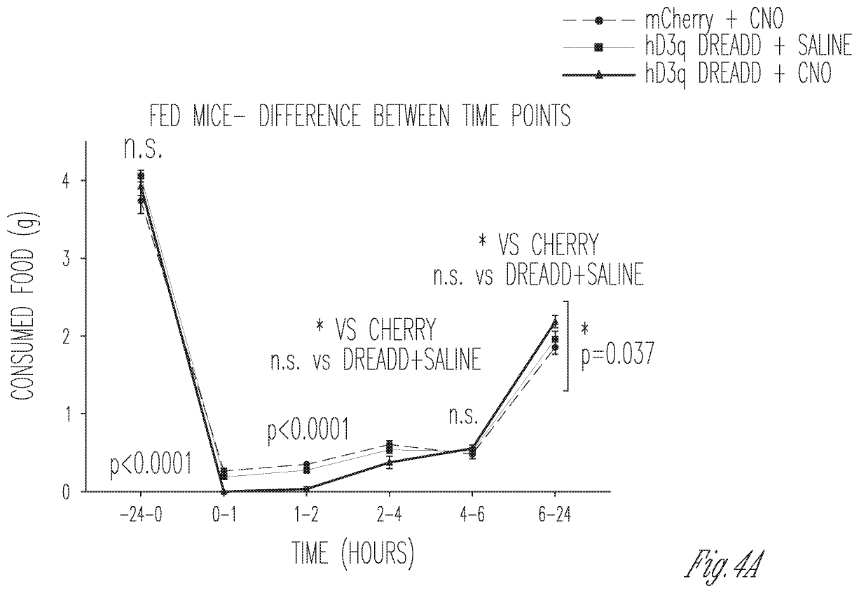

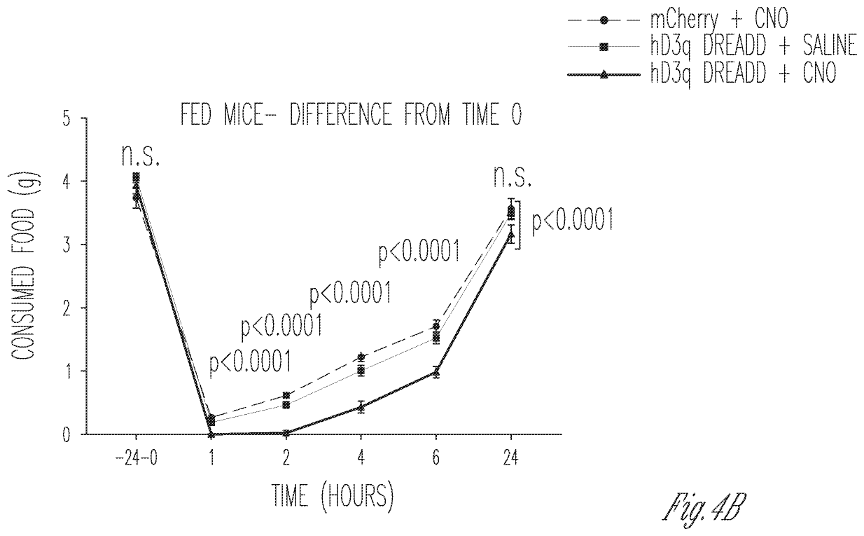

FIG. 3 is data of feeding behavior in fasted mice with 1 mg/kg CNO and FIG. 4 is data of feeding behavior in normally fed mice with 1 mg/kg CNO.

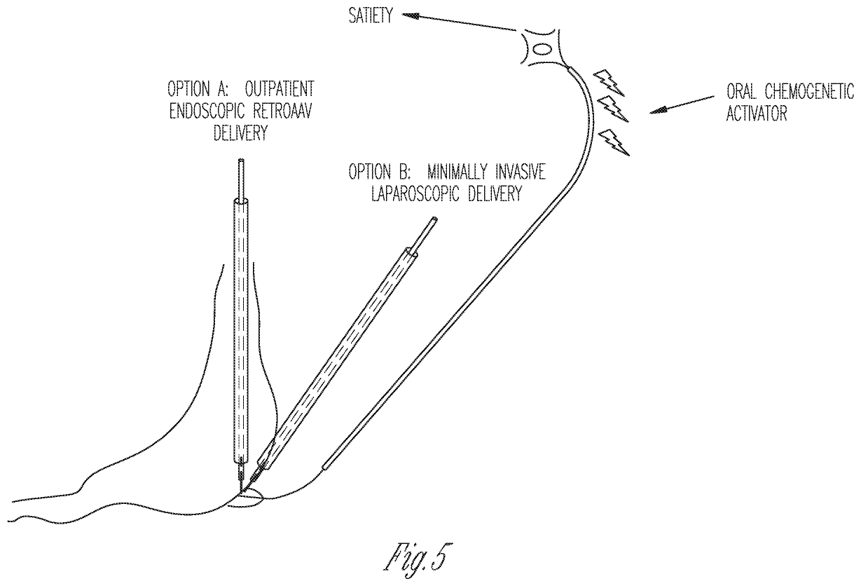

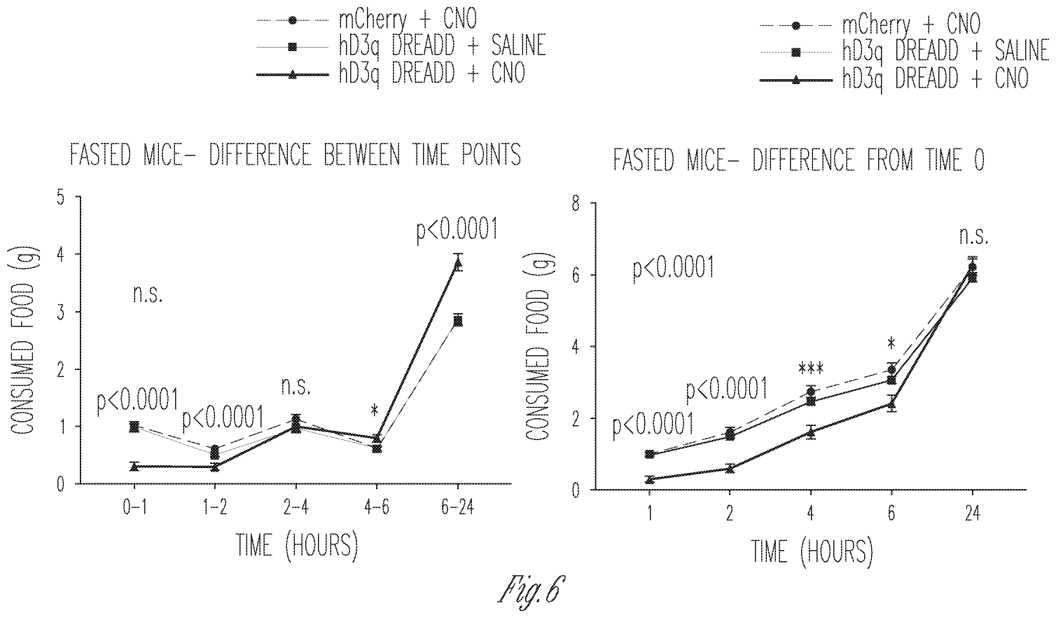

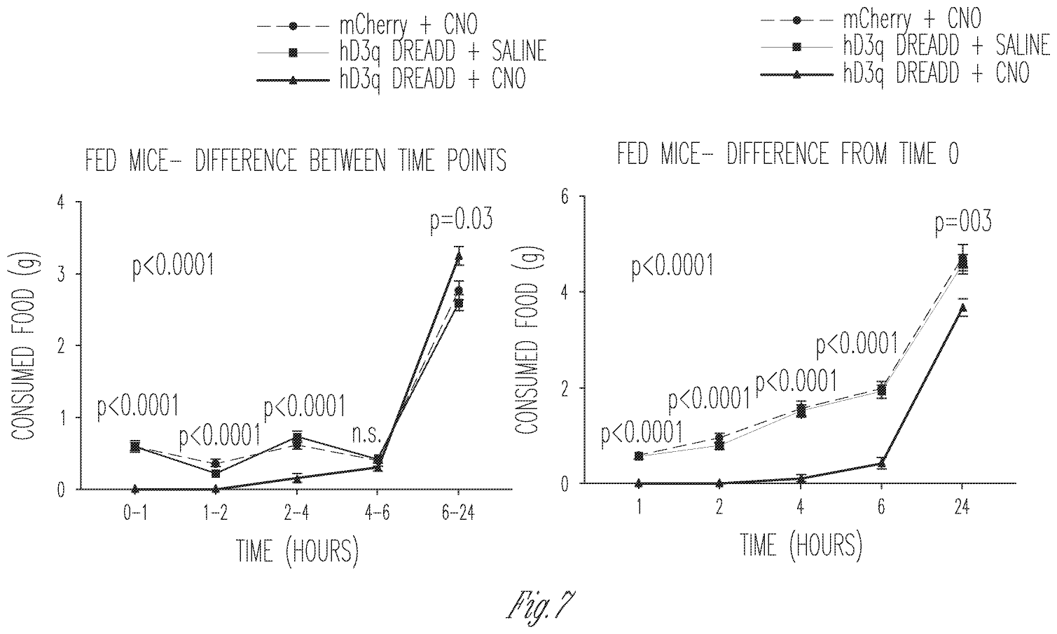

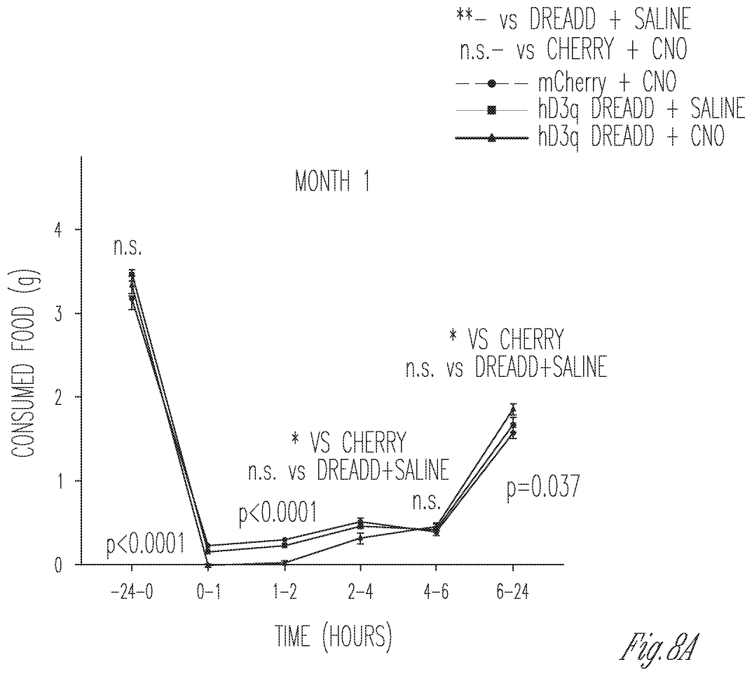

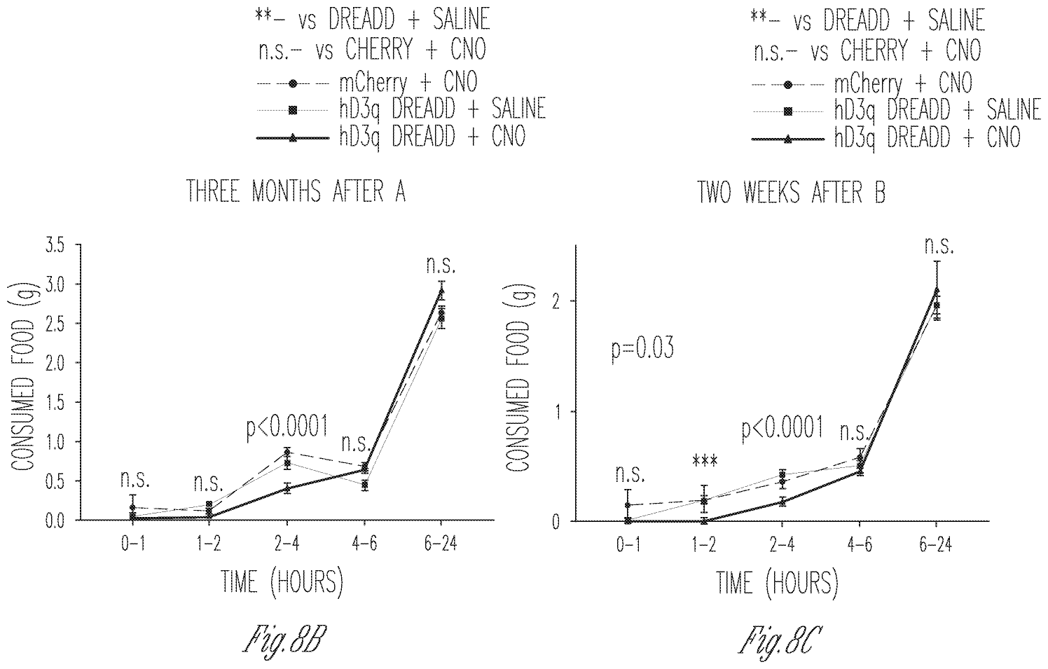

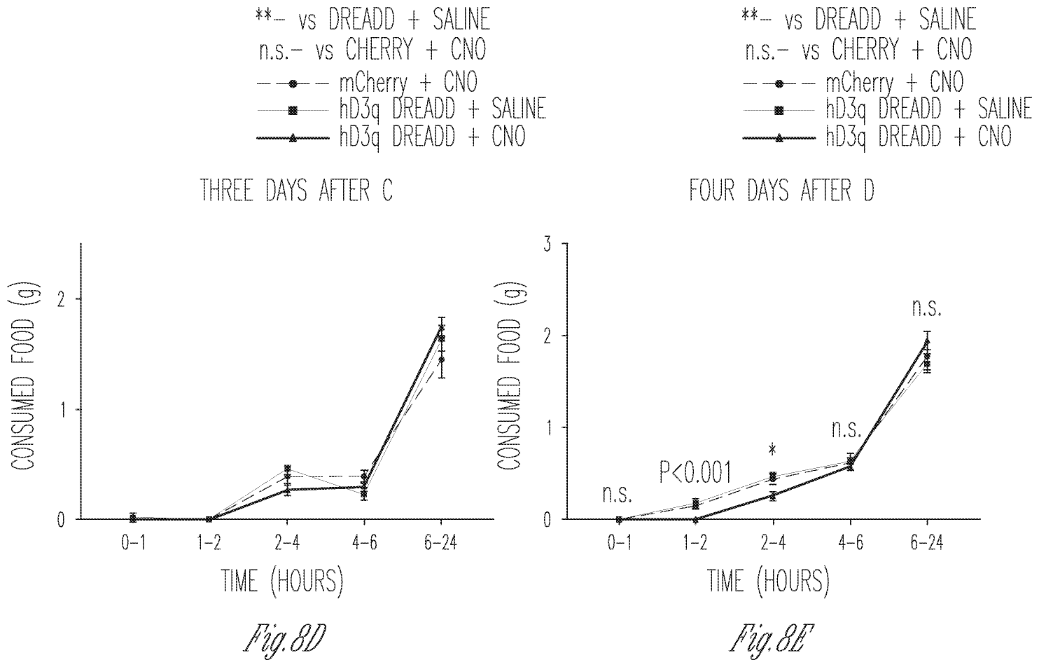

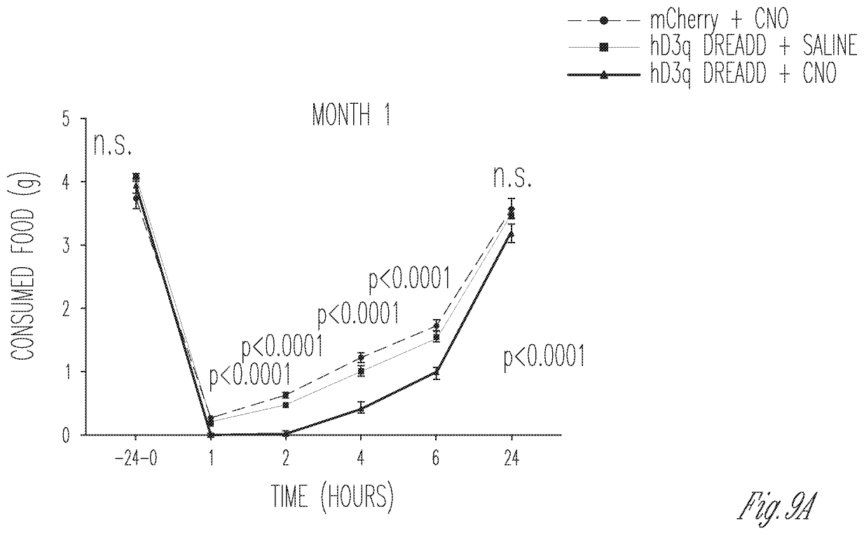

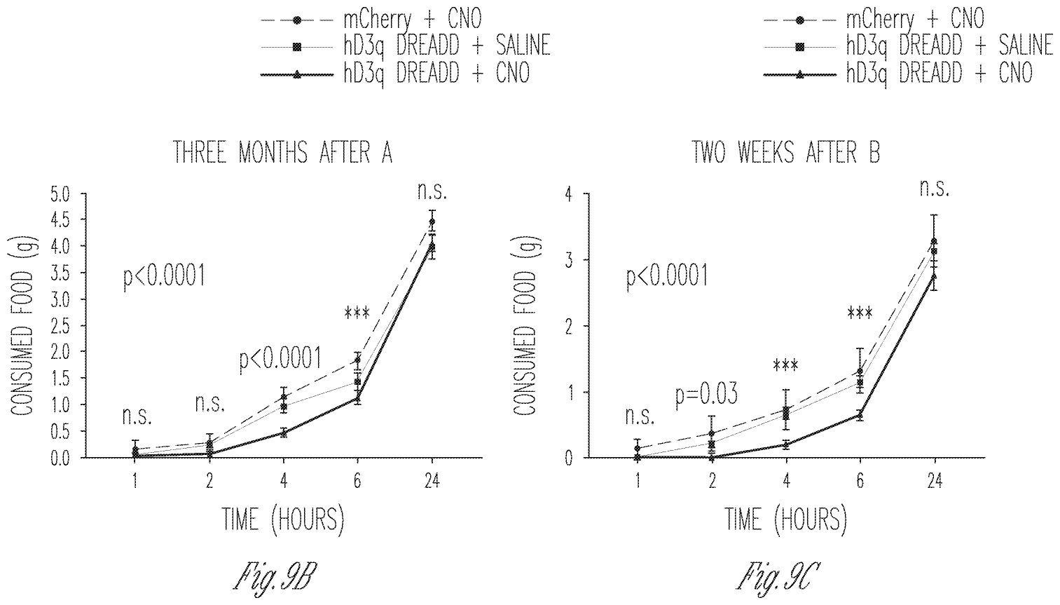

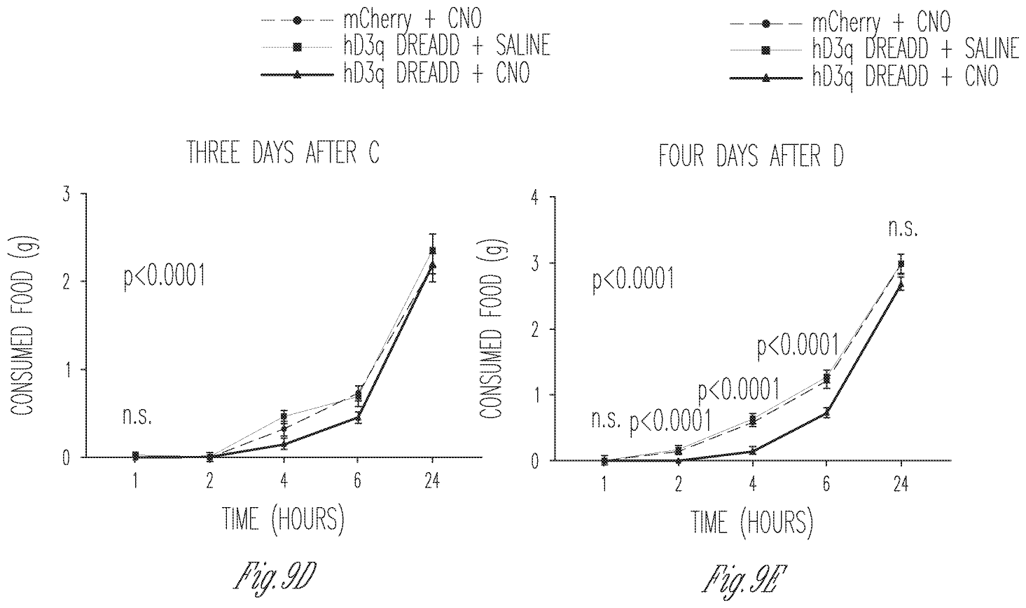

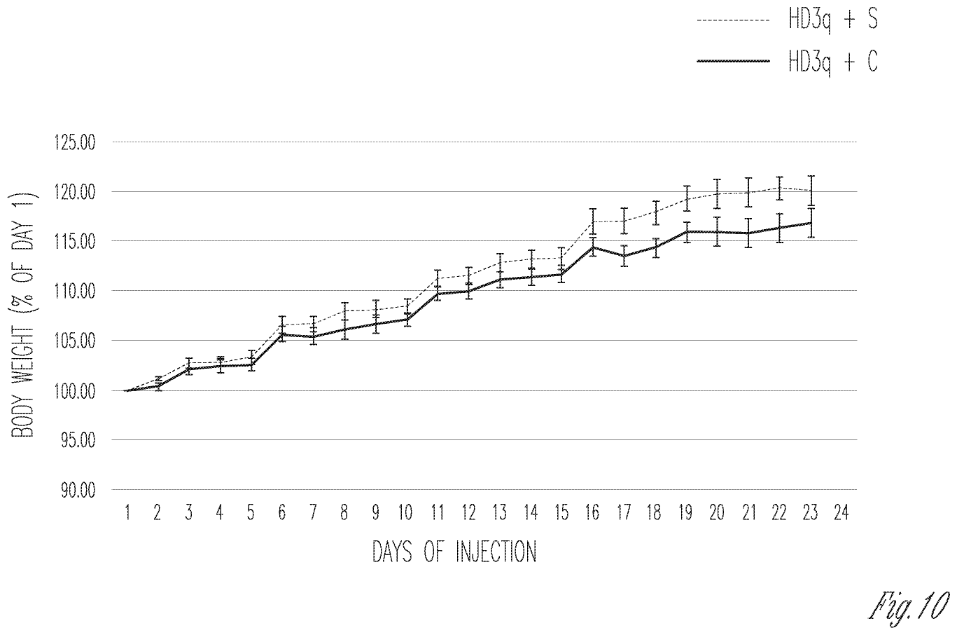

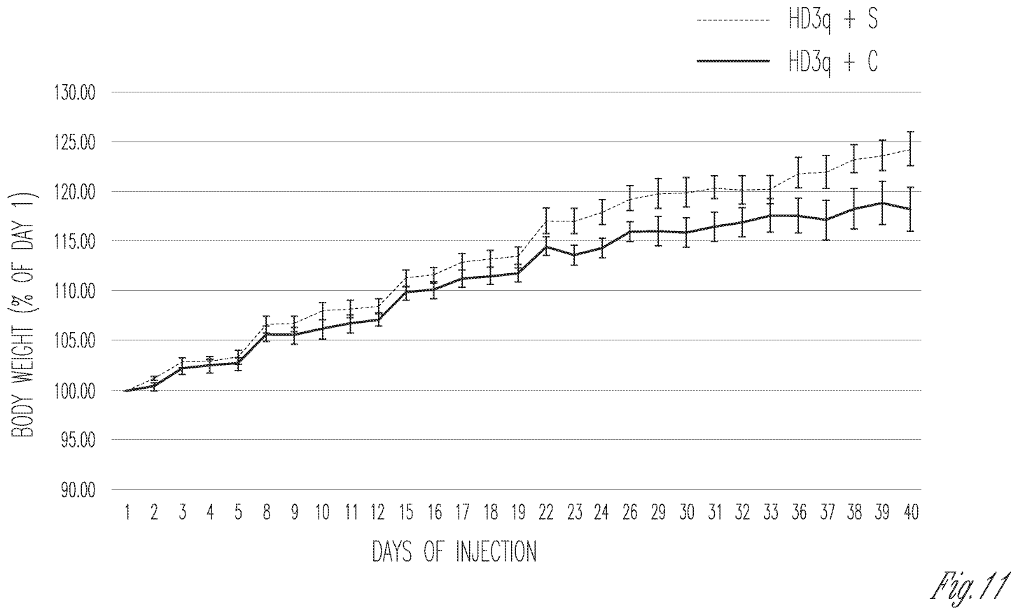

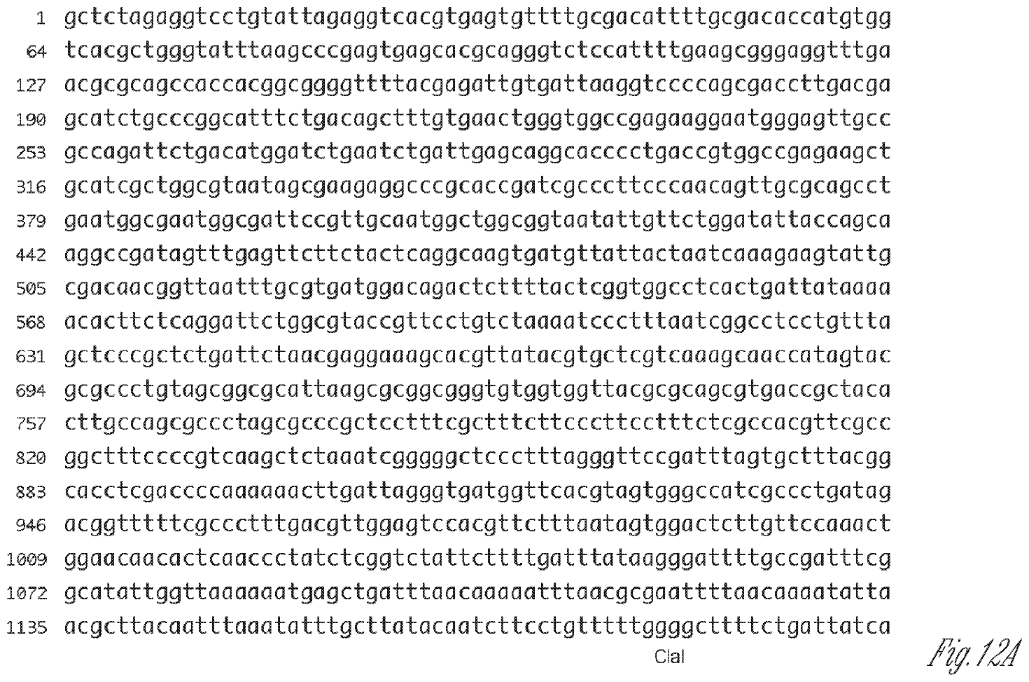

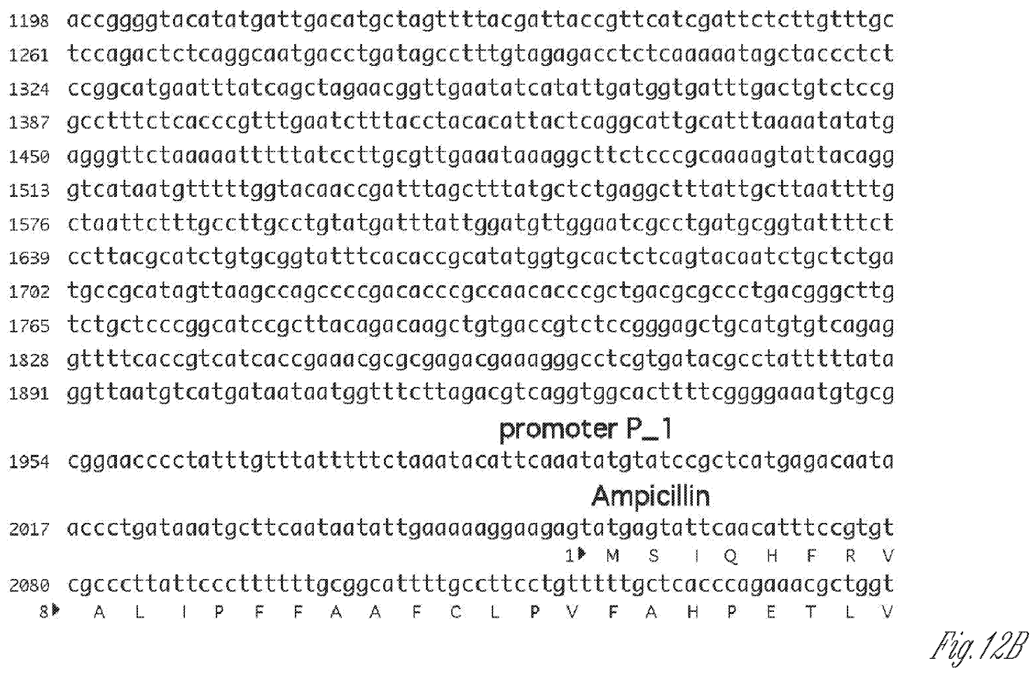

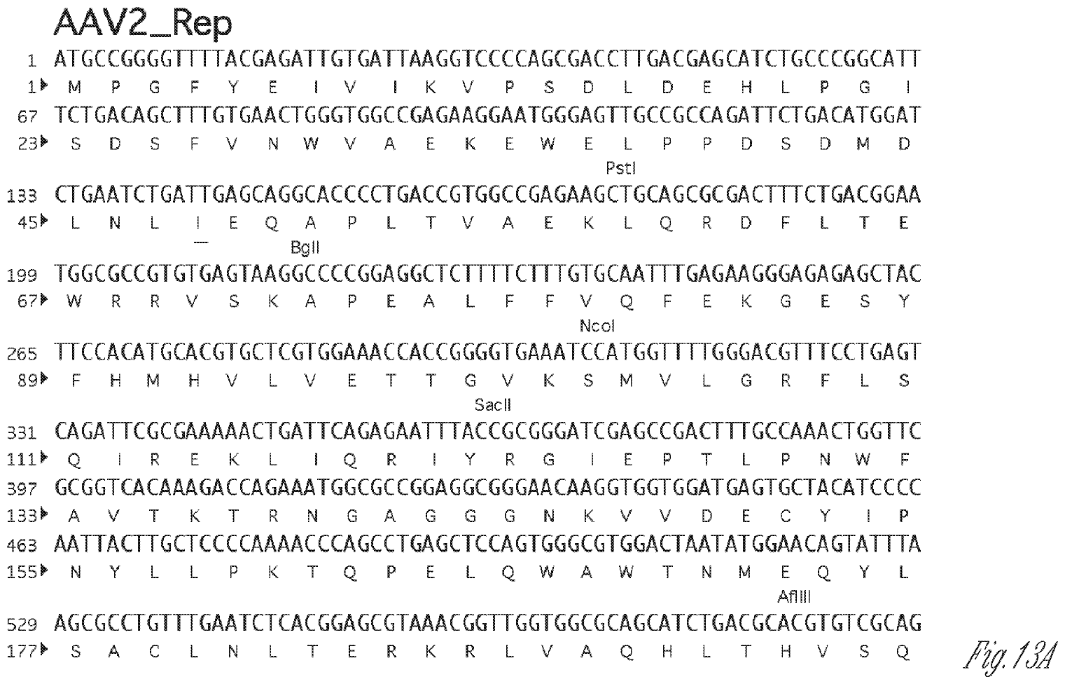

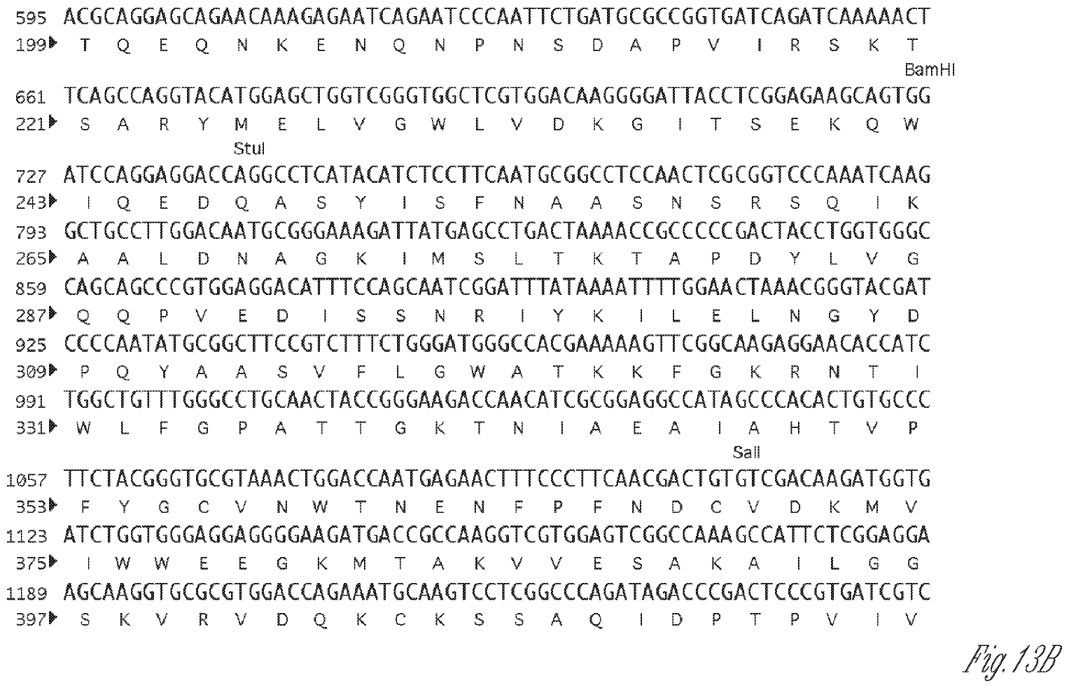

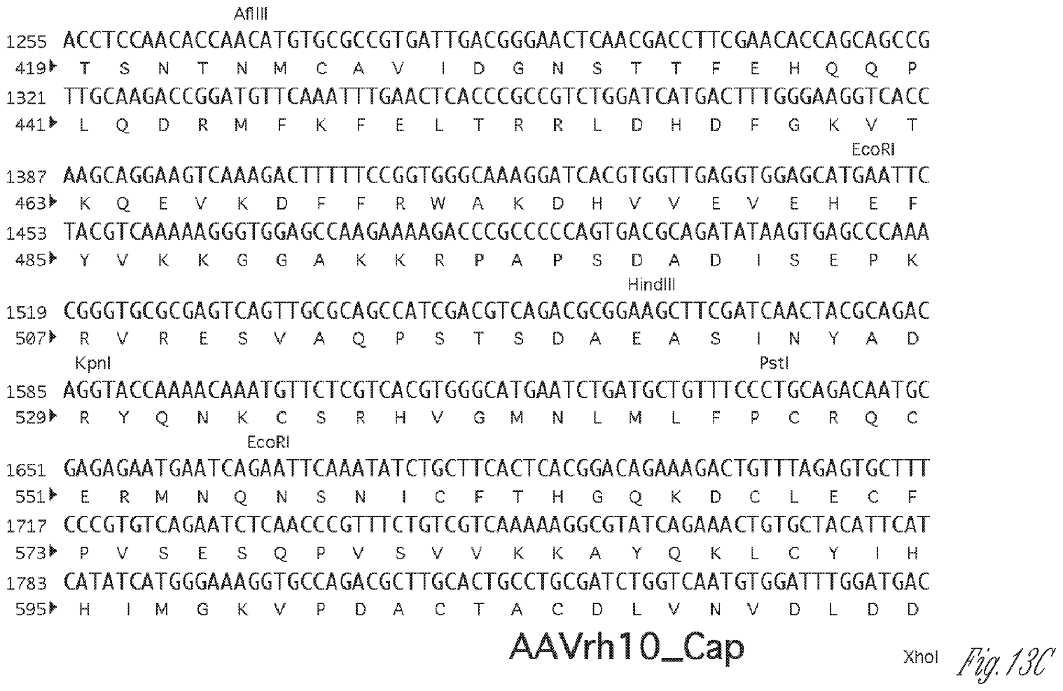

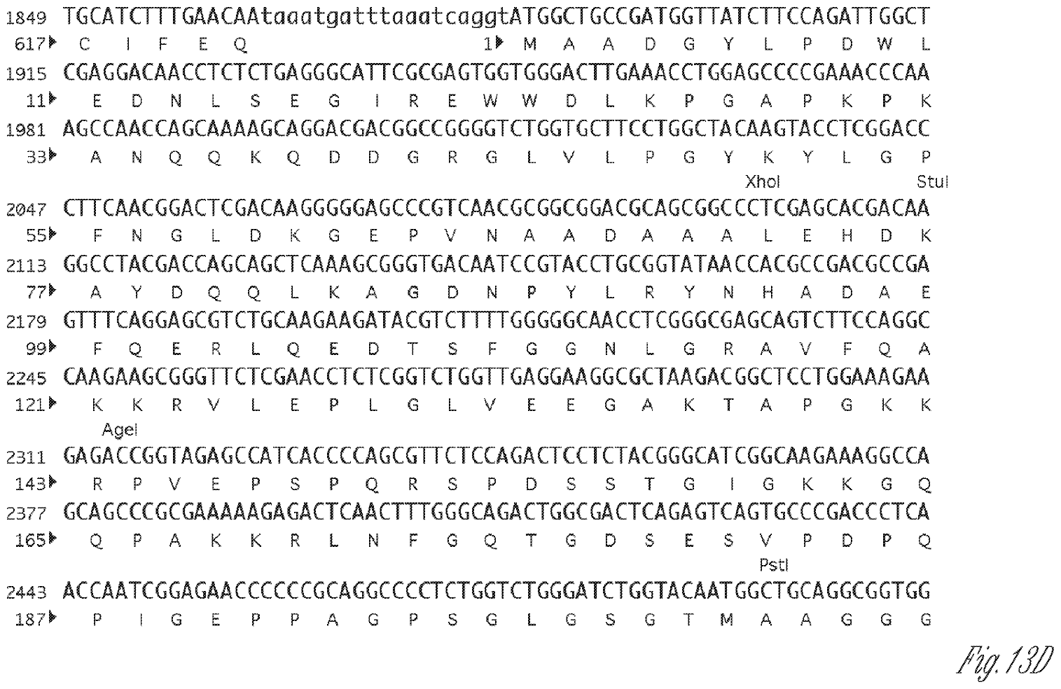

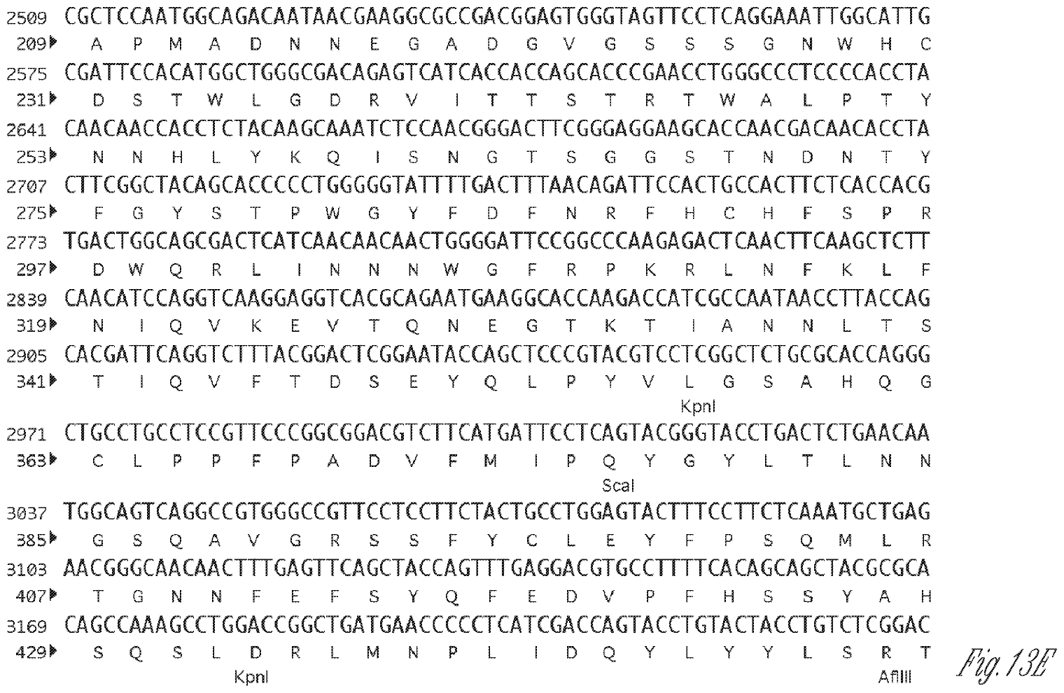

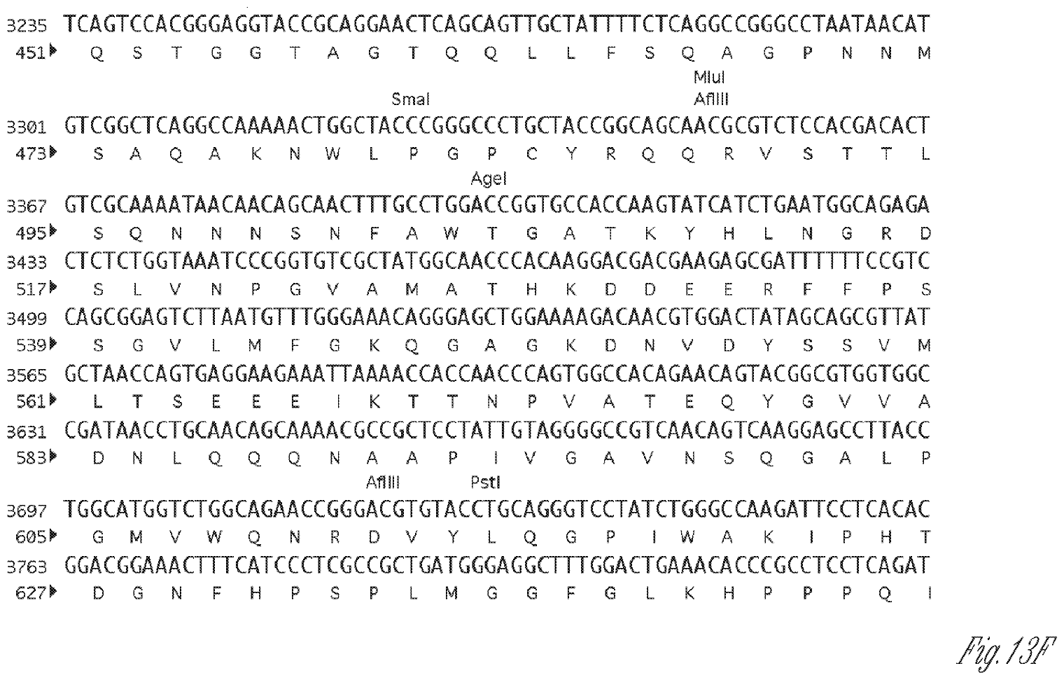

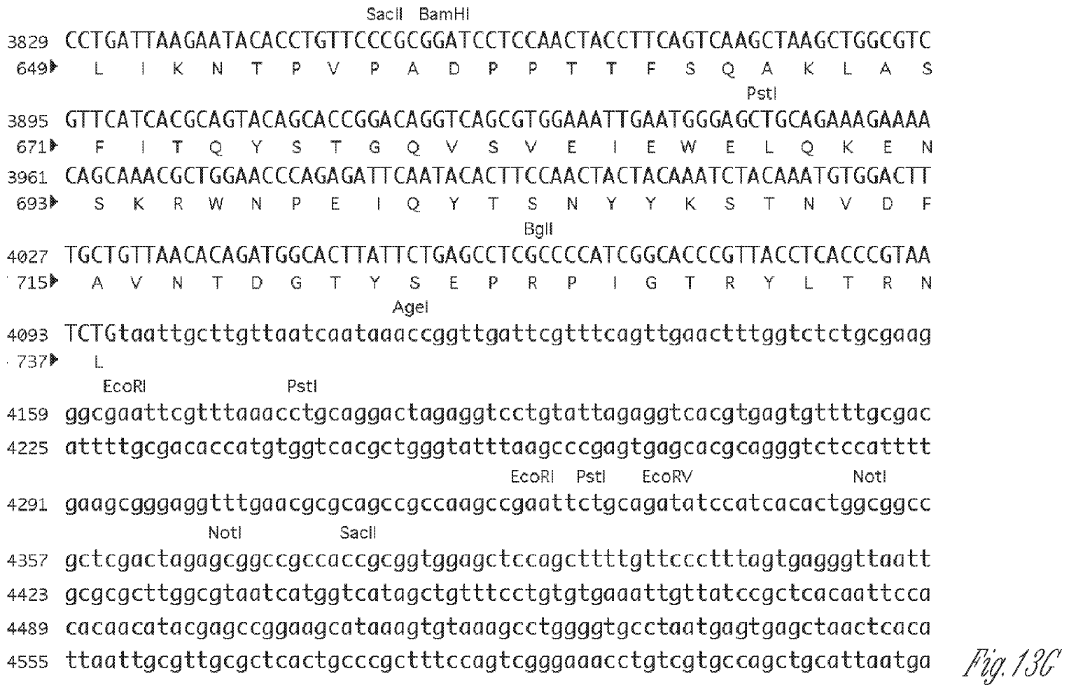

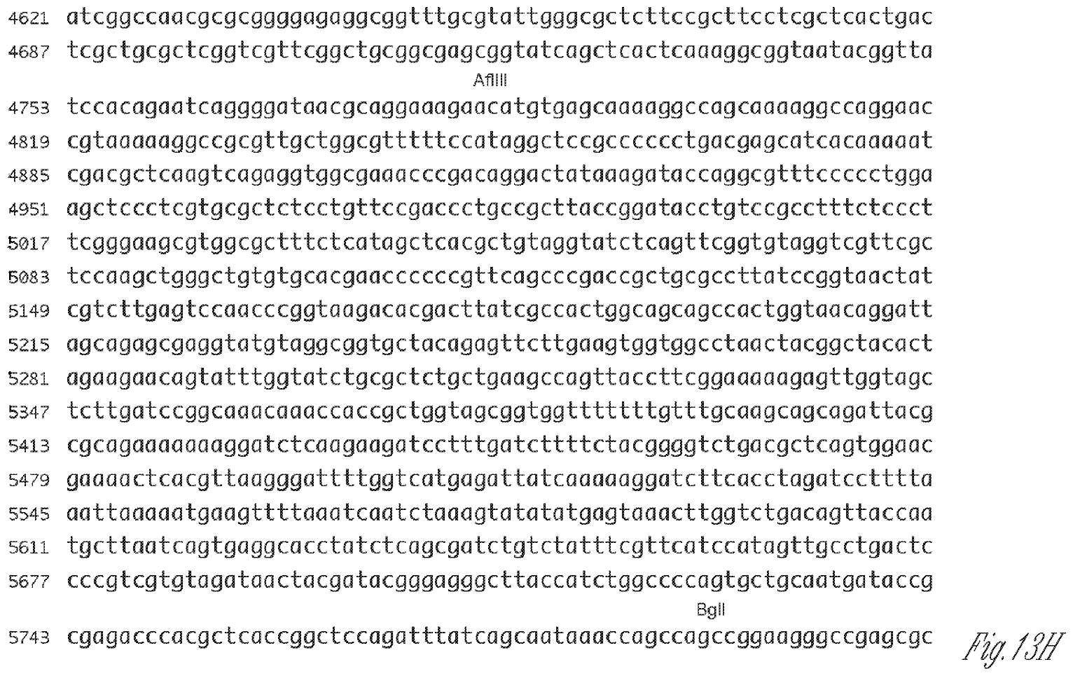

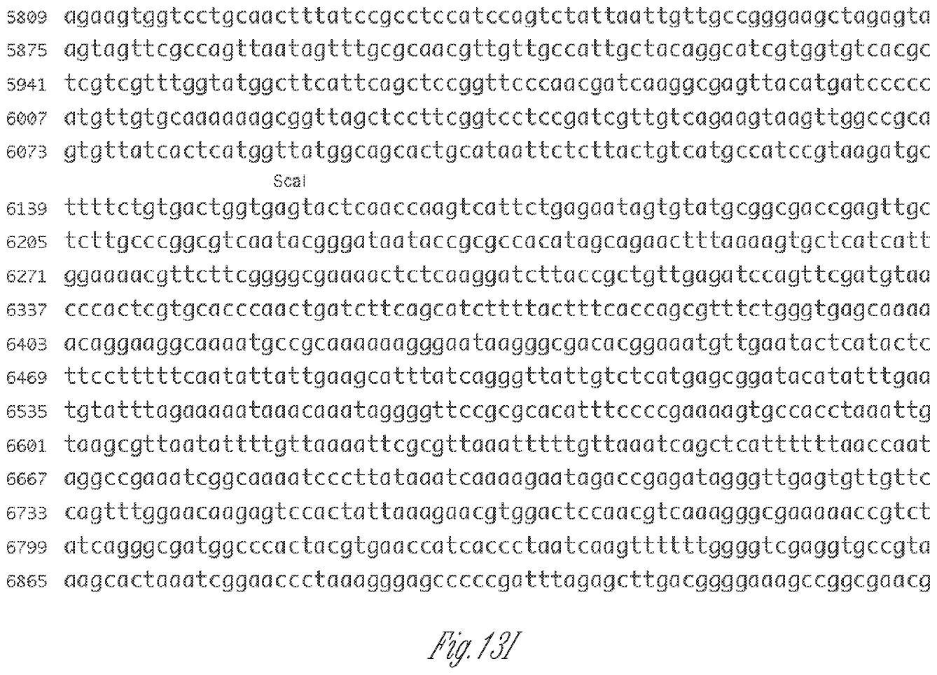

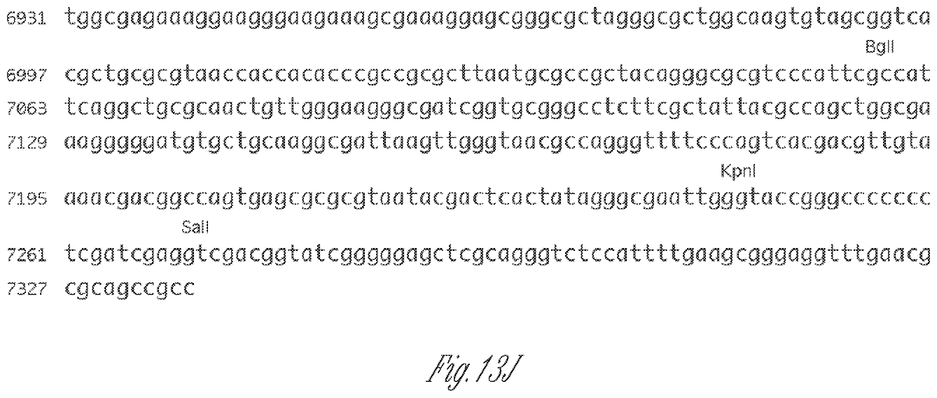

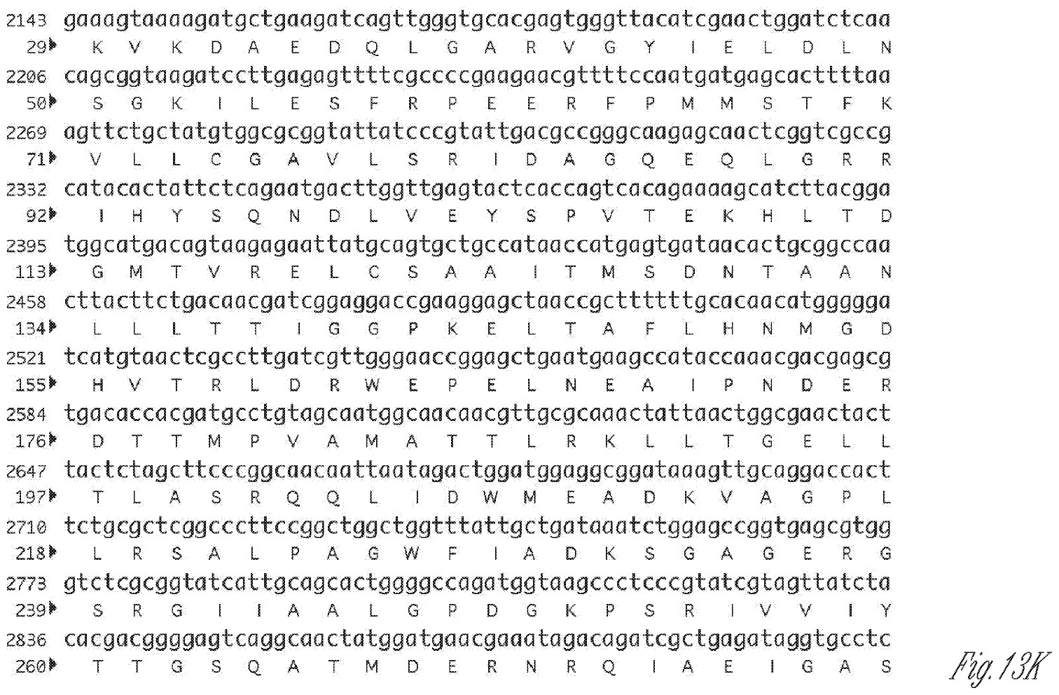

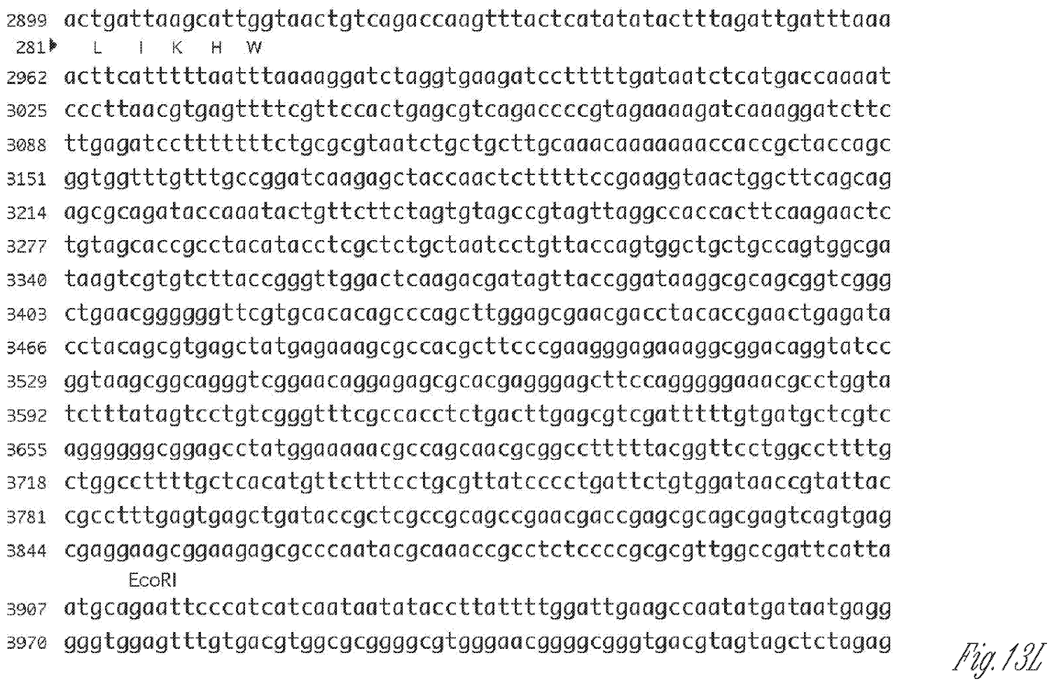

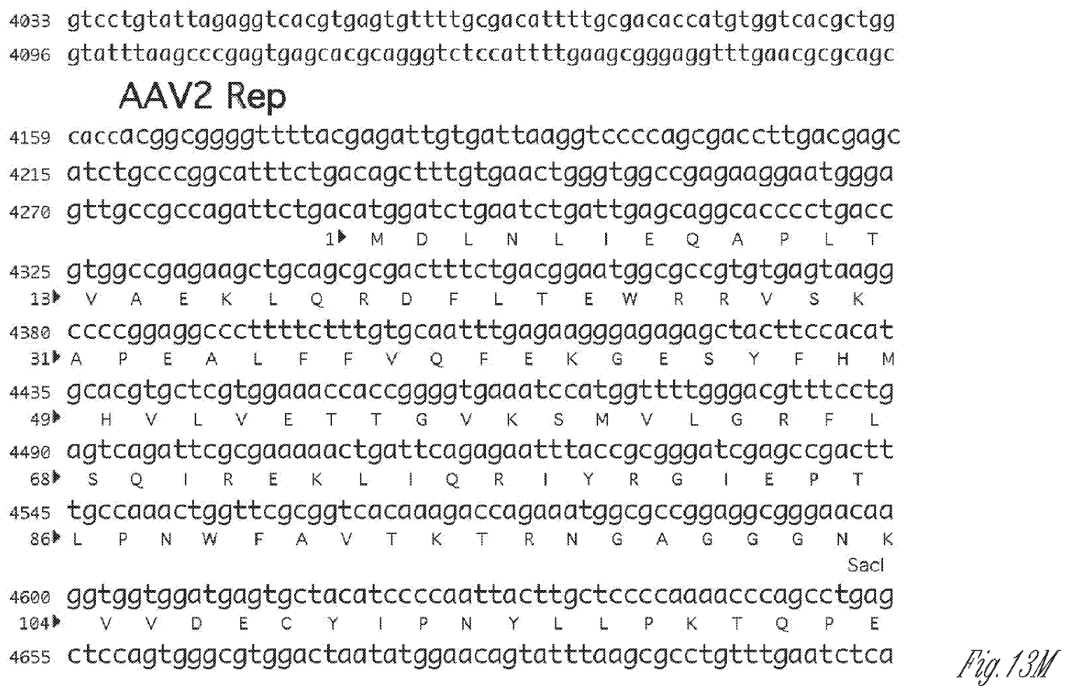

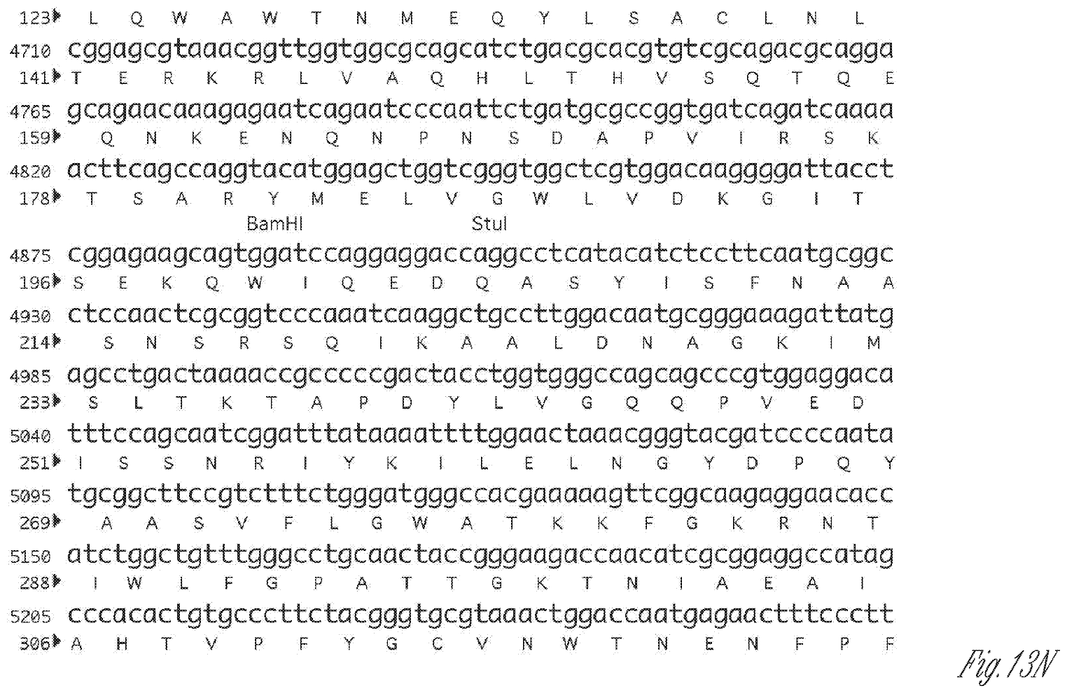

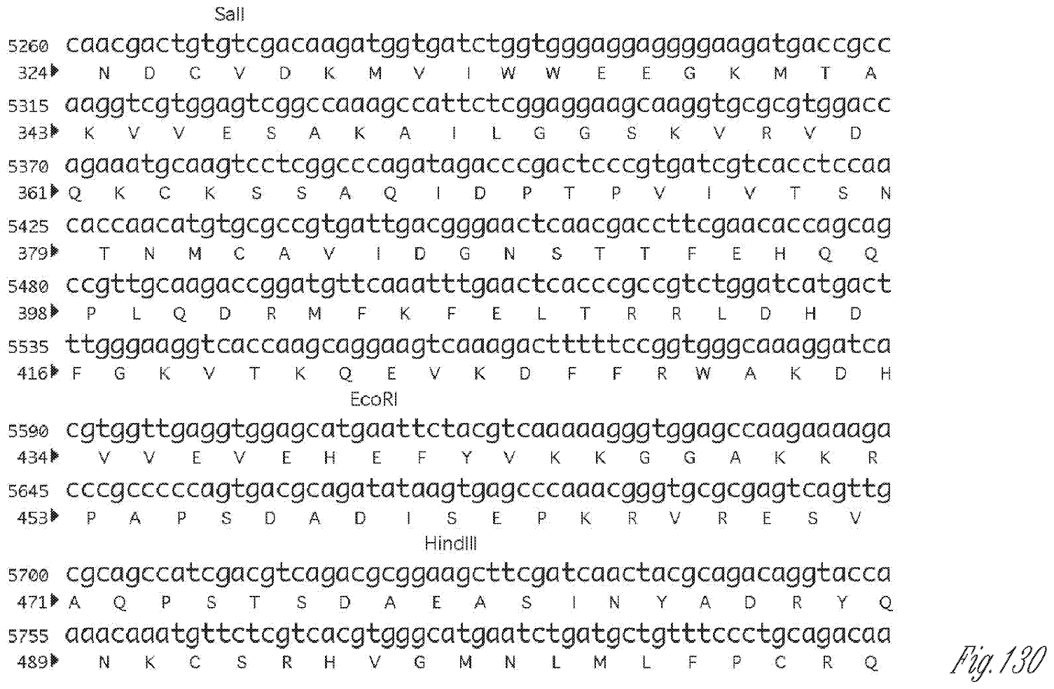

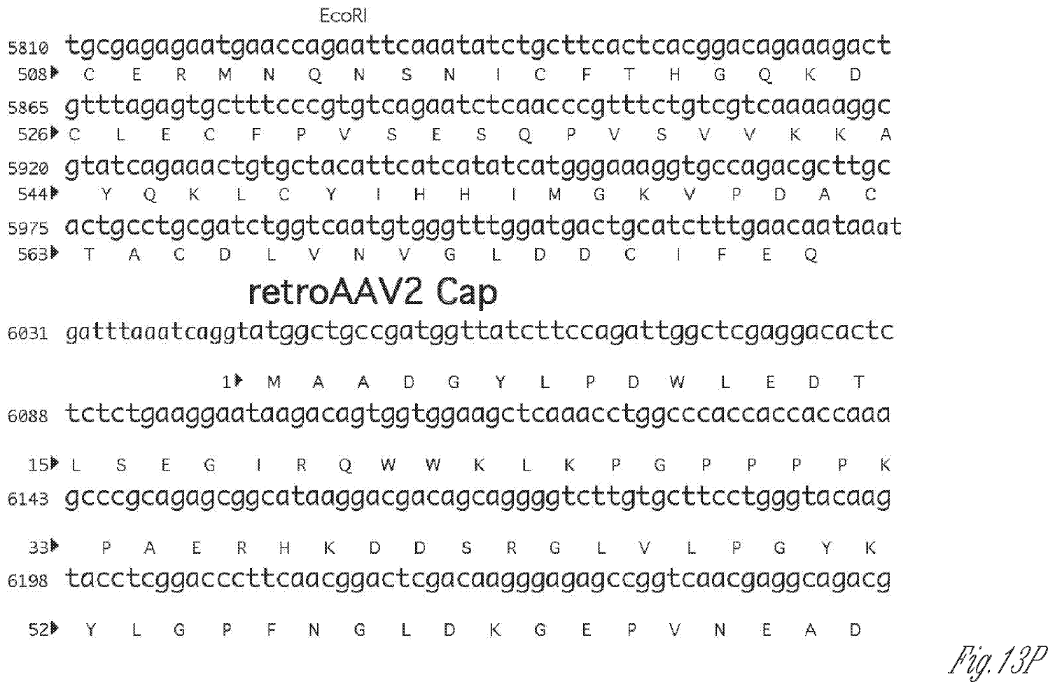

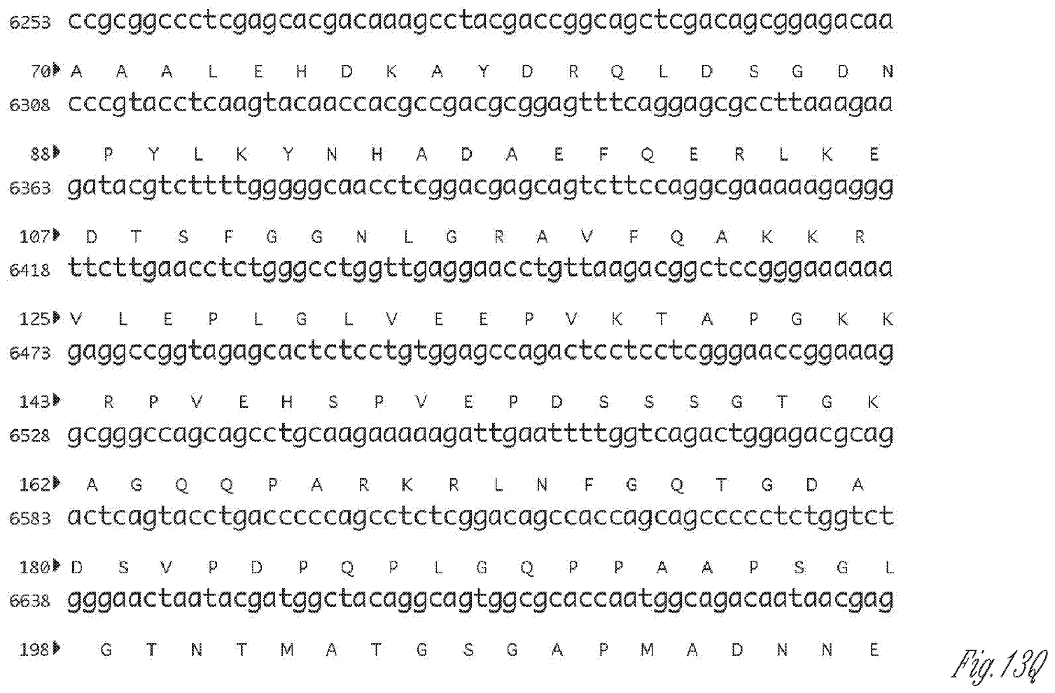

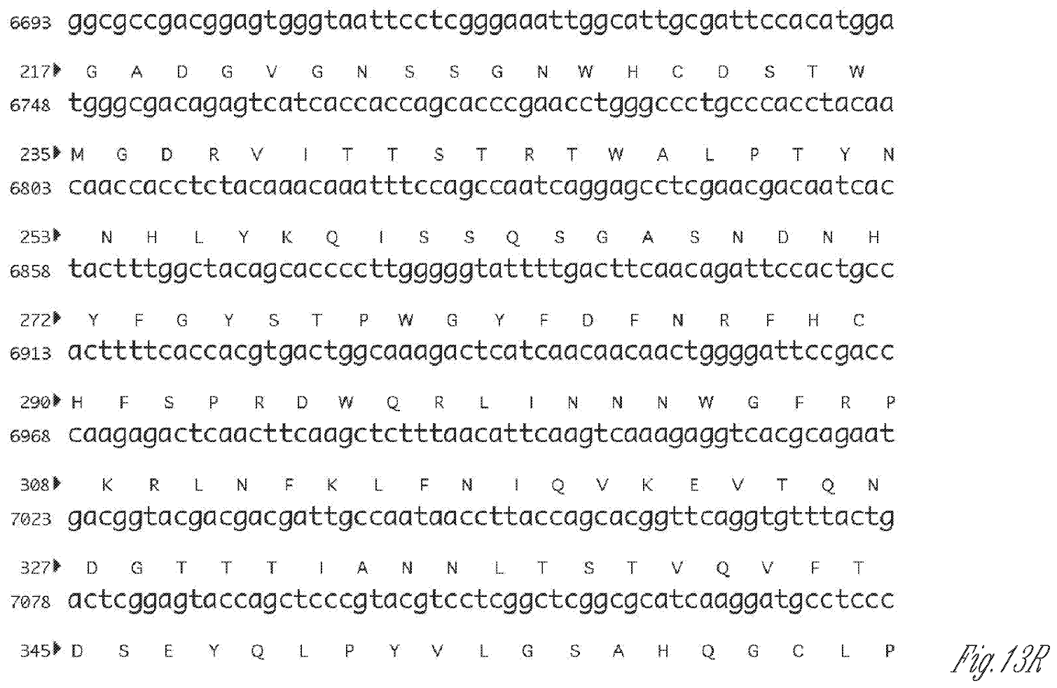

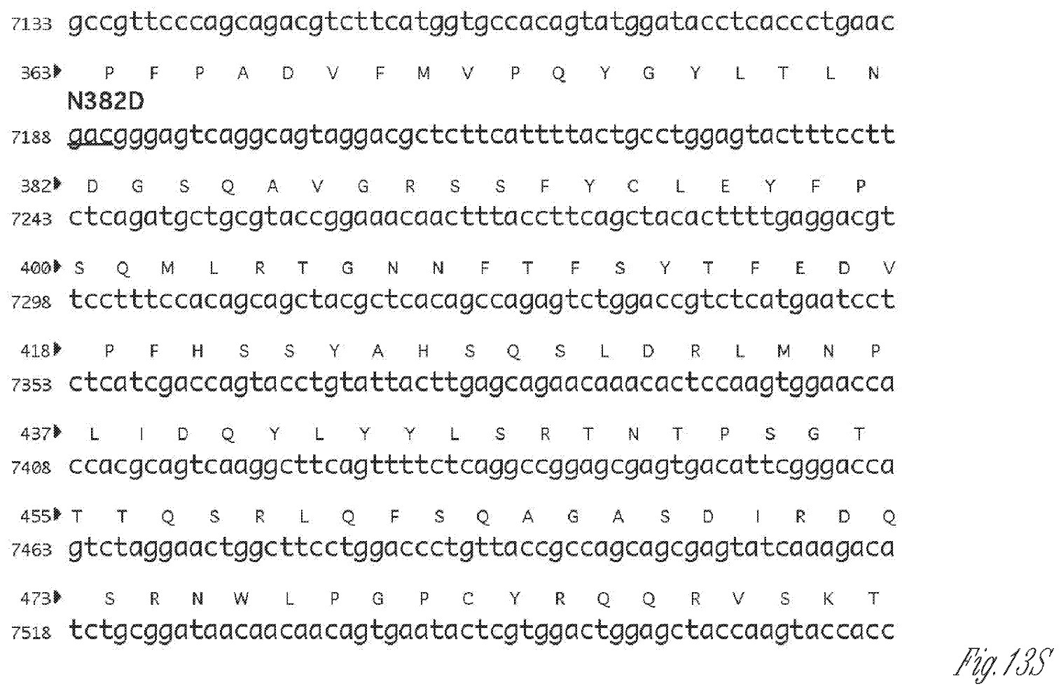

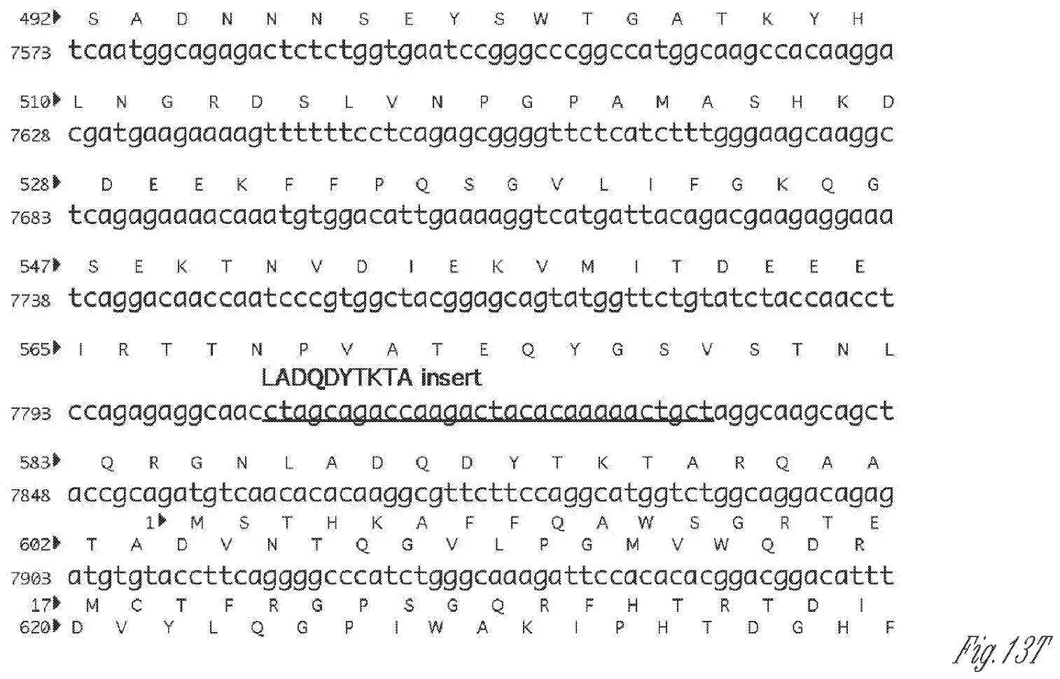

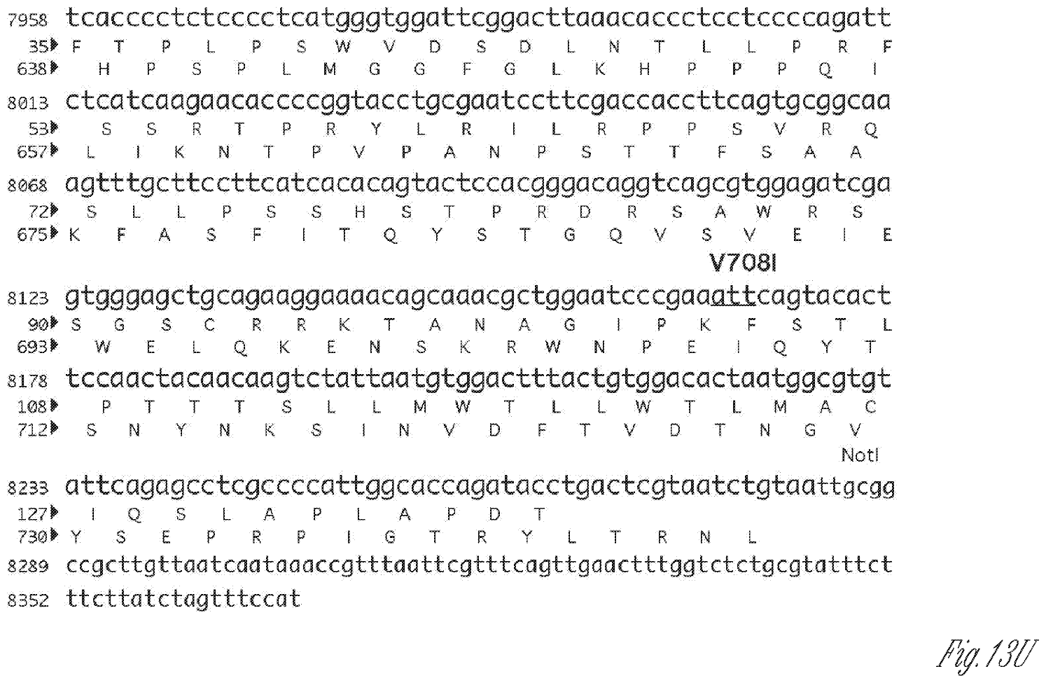

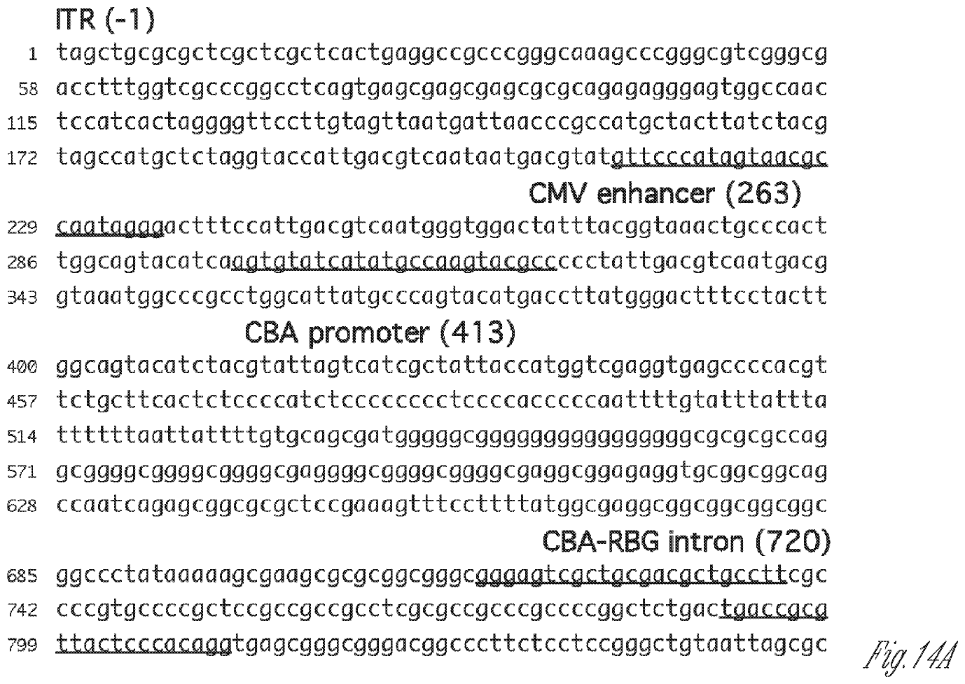

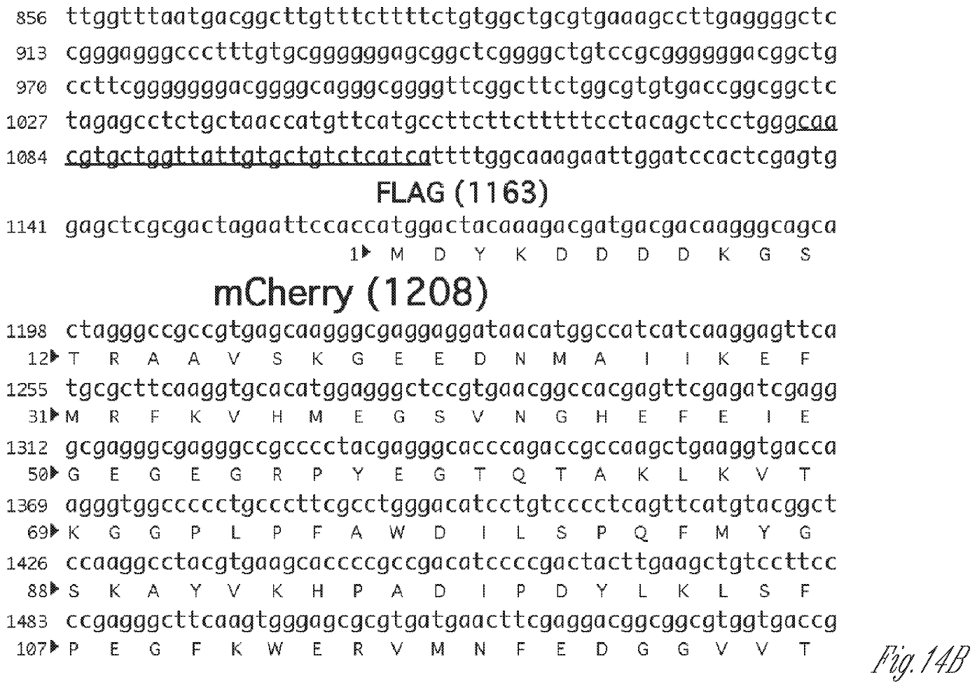

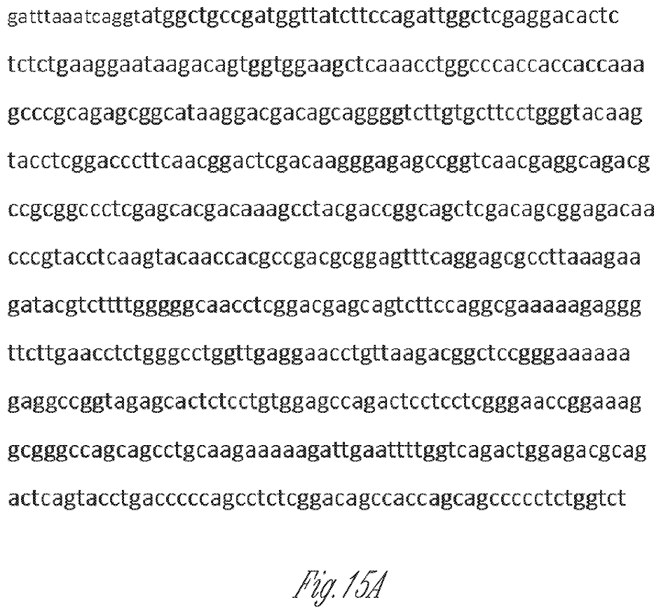

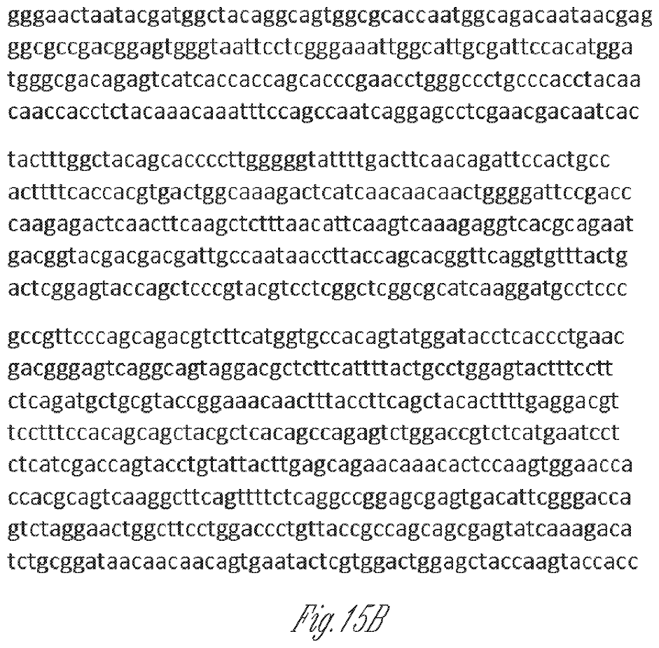

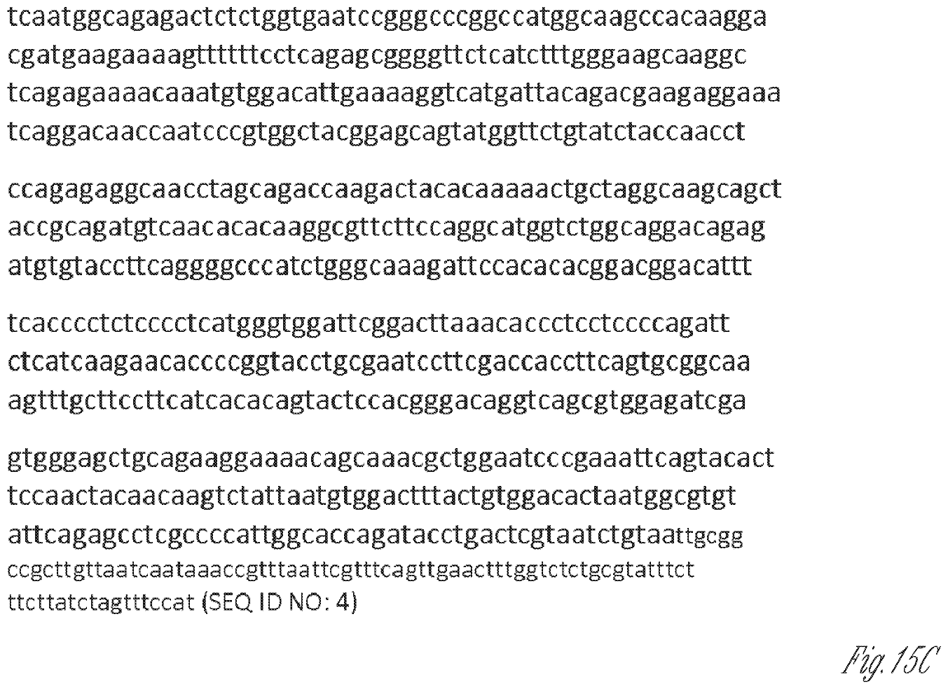

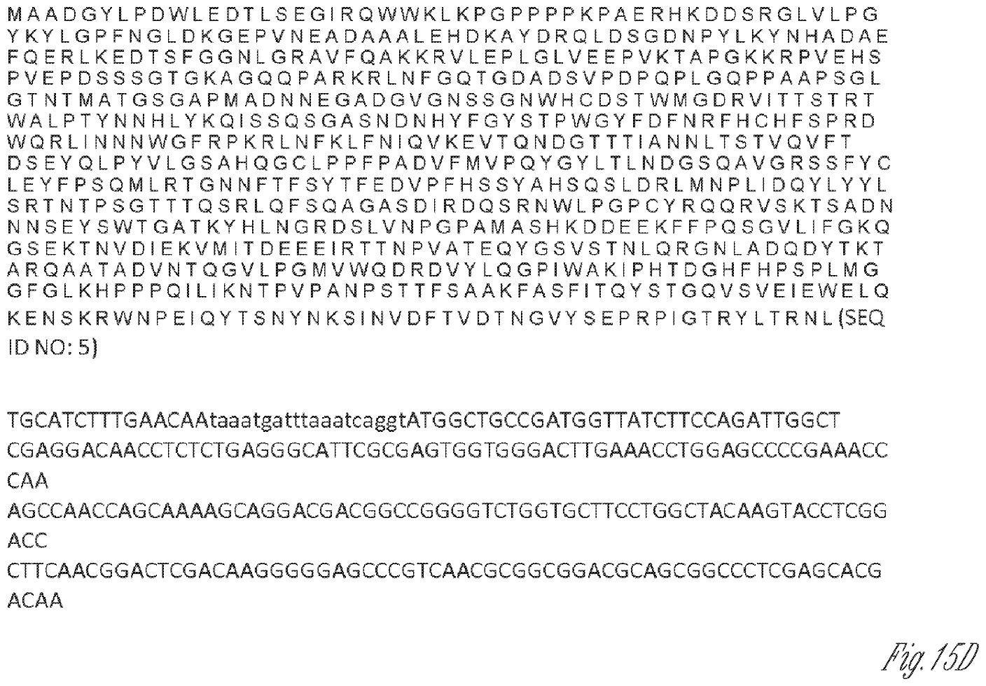

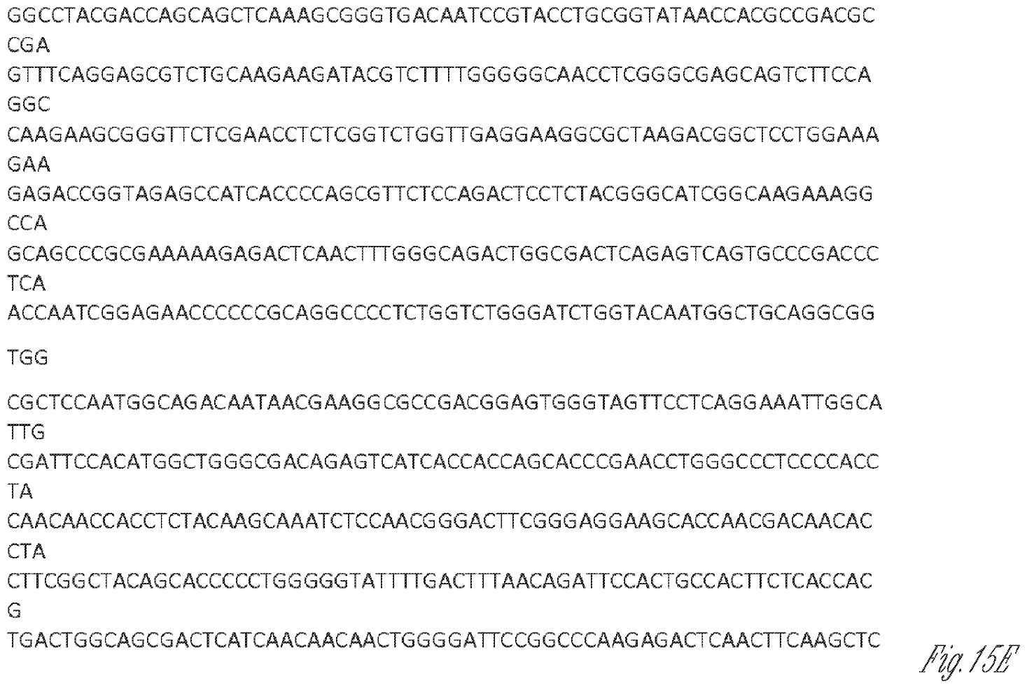

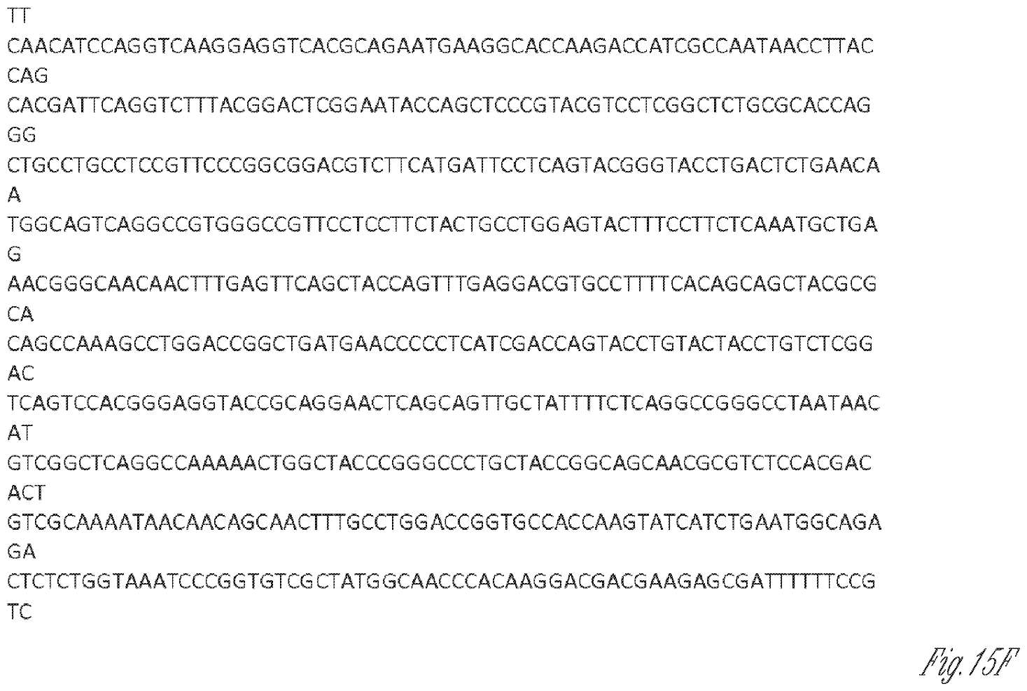

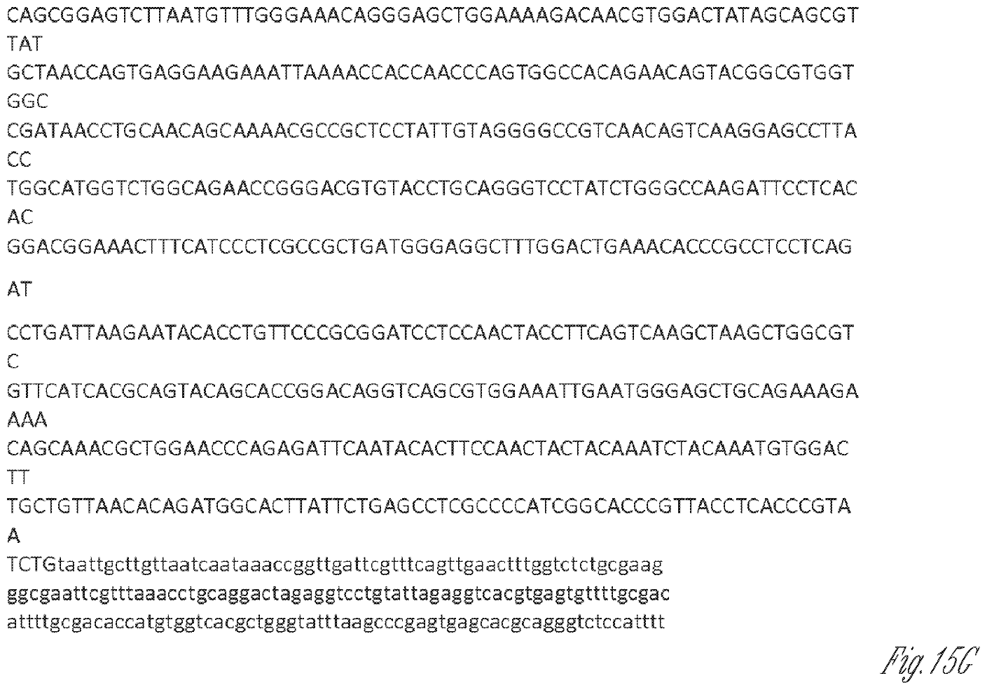

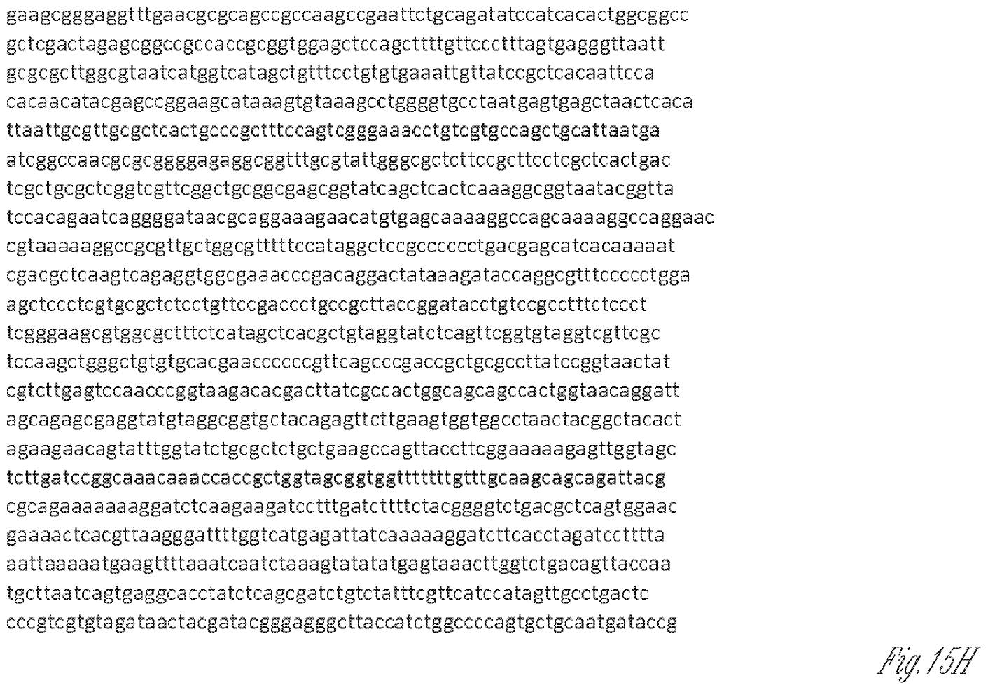

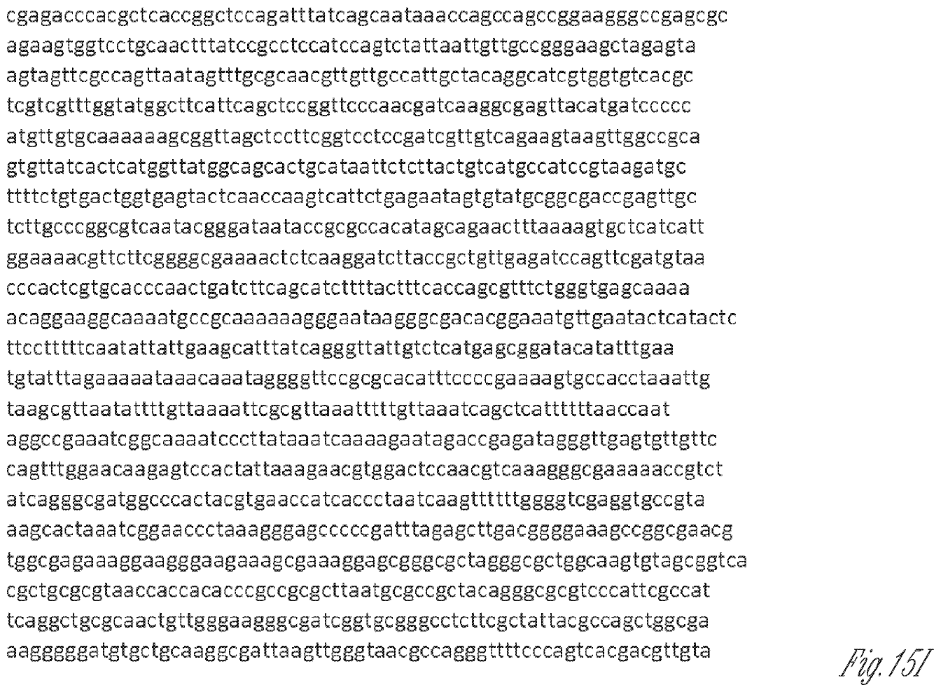

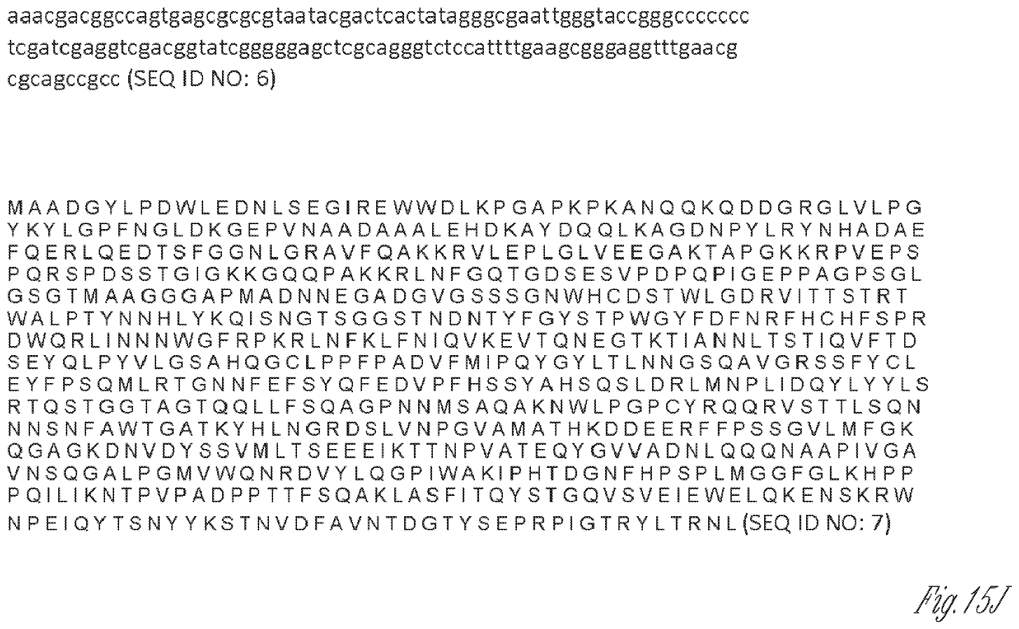

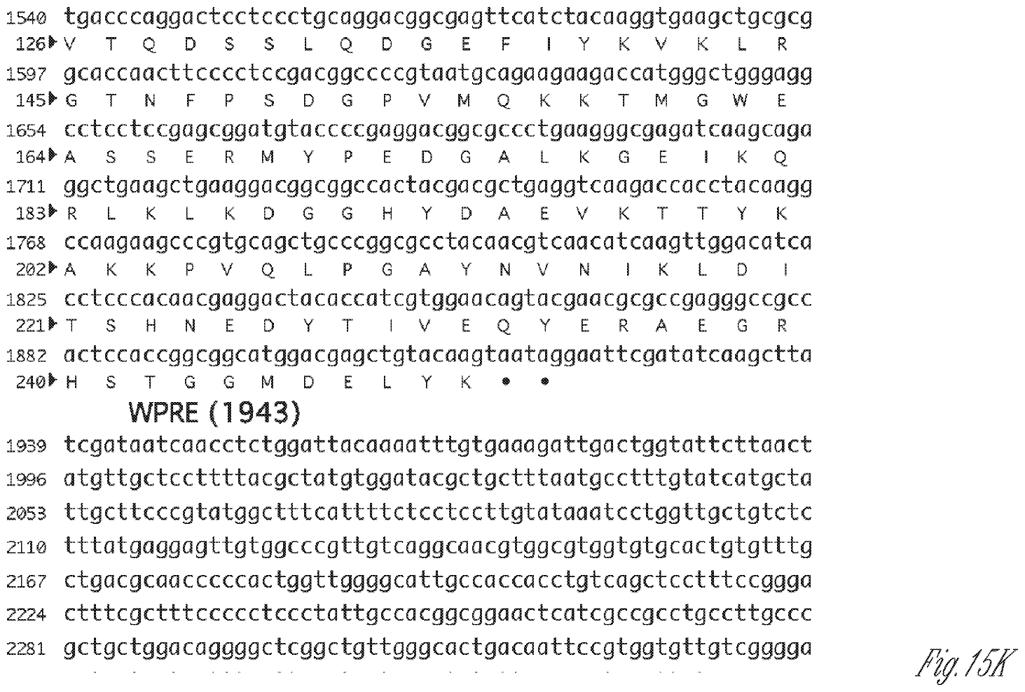

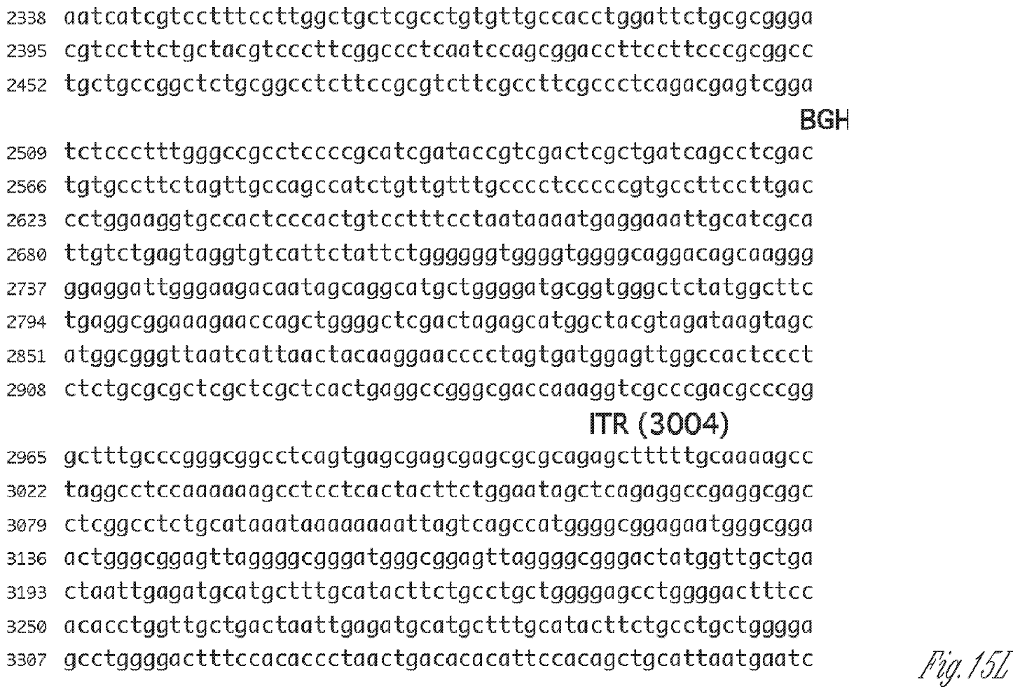

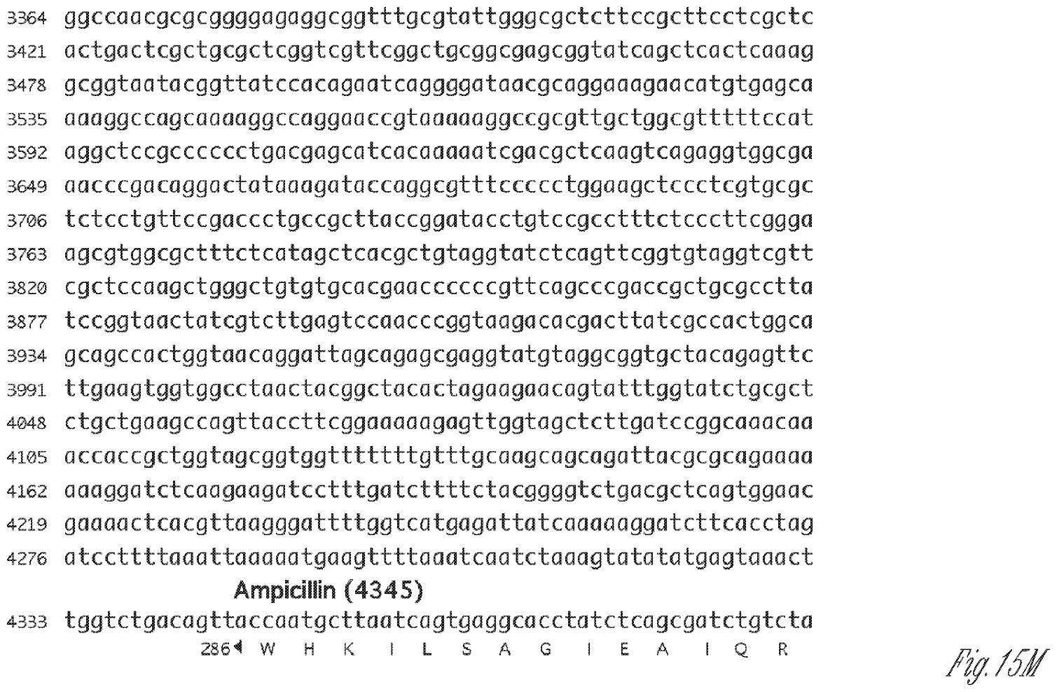

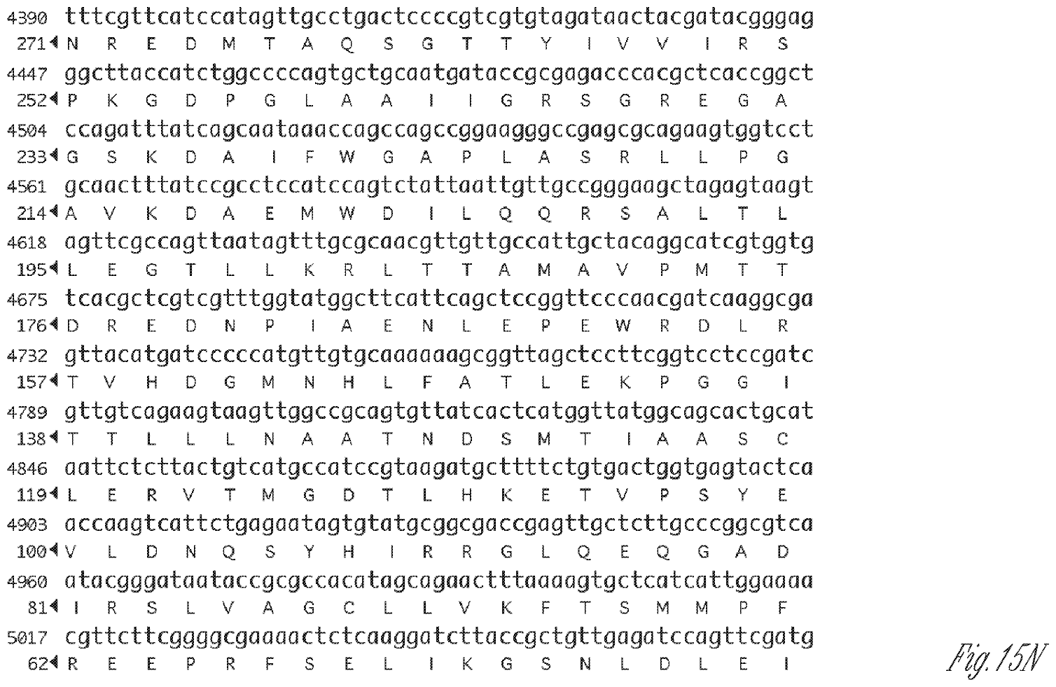

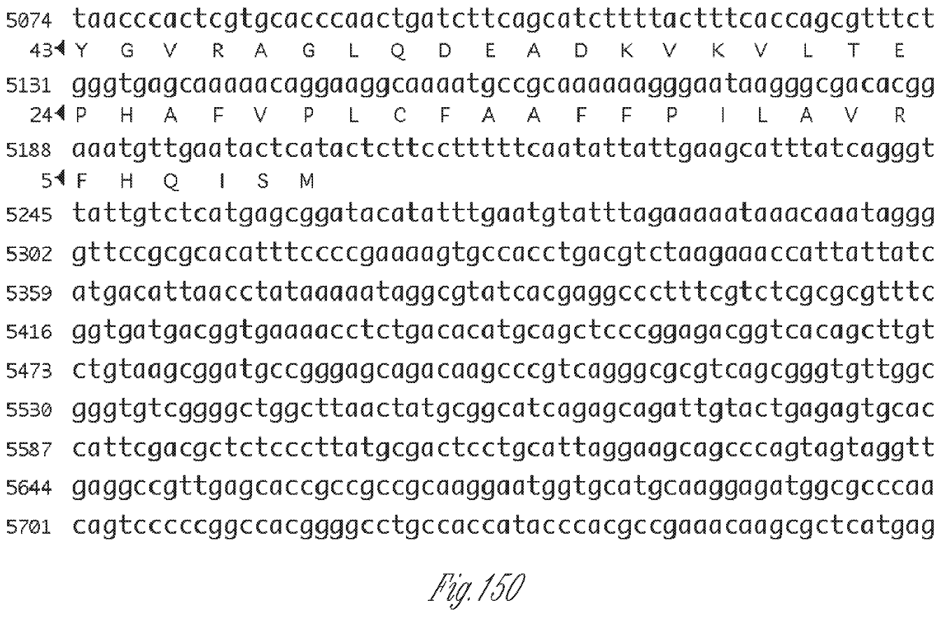

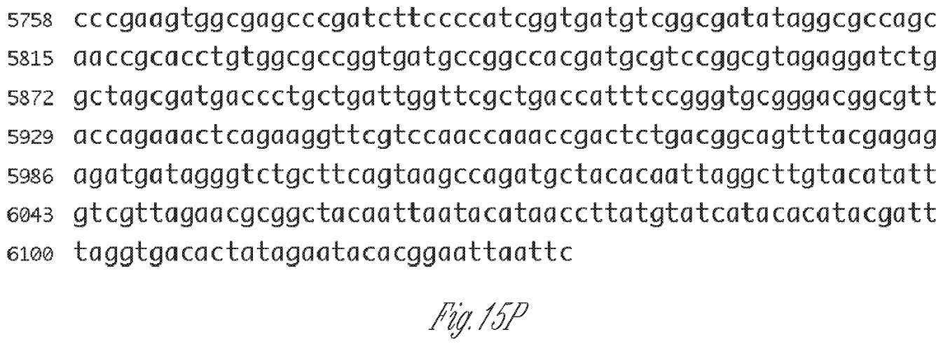

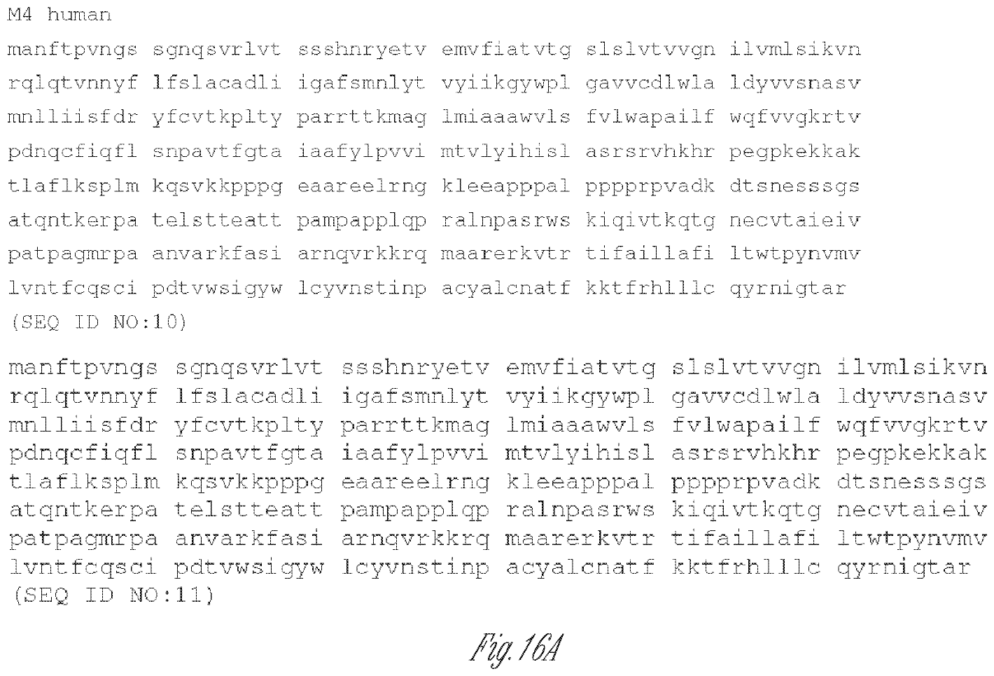

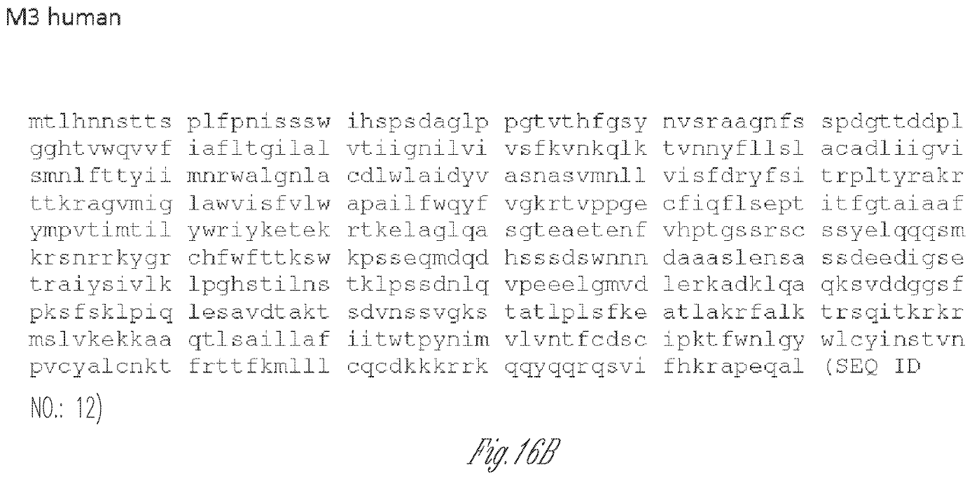

One approach to delivering a viral vector to control organ functions is shown in FIG. 5. For example, the composition having a rAAV encoding hM3Dq, see, e.g., SEQ ID Nos. 10 or 11 in FIG. 16 which is modified to DREADD hM3Dq, may be delivered endoscopically or laproscopically, e.g., to the greater curvature of the stomach which in one embodiment allows for virus delivery to the vagal afferent nerves. In some embodiment, administration may allow for delivery to vagal efferent nerves or motor neurons. In one embodiment, the composition is delivered to a visceral organ including but not limited to heart, liver, lung, adrenal, thyroid, pancreas, intestine, kidney, bladder, or spleen.

In one embodiment the chimeric AAV capsid comprises a ratio of 0.1:1, 0.5:1, 1:1, 1:3, 1:4, 1:5, 1:15, 1:20, 1:50, 1:100, 1:500 of one AAV serotype, e.g., AAV2, to another AAV serotype, e.g., AAVrh10 capsid.

### Example 2

The cough reflex normally is important for expelling potential obstructive or infectious agents within the airways of the lung. Intractable cough is usually treated by attempting to address the underlying cause of the cough, such as gastric reflux, chronic inflammation from asthma or chronic obstructive pulmonary disease or malignancy. For many people, however, chronic cough is the primary problem without a clear ongoing irritant or stimulant which can be addressed. This is similar to intractable pain, which can be due to an underlying pathology that needs to be reversed but often the pain itself needs to be addressed as there is no abnormality that can be reversed. For patients with intractable cough without a reversible pathology, there are few treatment options. Narcotics can be used to try to suppress the cough, but these have major morbidity particularly with chronic use.

In order to suppress cough in an animal model, retroAAV2/rh10 expressing the inhibitory DREADD hM4Di (1×10¹² p/ml) (see, e.g., SEQ ID NO:12 for M4 in FIG. 16 which is modified to DREADD hM4Di) is aerosolized and sprayed into the trachea and upper airway of guinea pigs. The guinea pig is utilized because they exhibit a robust cough reflex, while rats and mice do not have an effective cough reflex. Six weeks following exposure, histological assessment using immunostaining confirms expression of the inhibitory opsin within a subpopulation of neurons within the nodose ganglion which provide sensation to the trachea and upper airway. A second group of guinea pigs are then divided into two cohorts. One cohort again receives the same retroAAV2/rh10.hM4Di aerosolized vector sprayed into the upper airway, while the second cohort receives retroAAV2/rh10 expressing mCherry as a negative control. Six weeks later, animals are placed into a closed plexiglass chamber. The chamber contains a pressure monitor to assay changes in air pressure within the chamber due to cough, and also contains a microphone that can capture the sound of a cough. These permit continuous monitoring of the frequency, overall number and intensity of coughs, and the monitors are time locked to confirm that a true cough occurs when both the sound and pressure change match. To induce coughing, the chamber is then filled with nebulized 2M citric acid at a rate of 5 L/min, which is sufficiently dilute that it does not cause permanent injury to the animal but will irritate the airway and cause coughing. Both cohorts are then exposed to the acid gas and the number, frequency and intensity of coughs in both groups are evaluated over 10 minutes to confirm that there is no difference between groups at baseline. A third group of untreated guinea pigs are exposed to the citric acid chamber to confirm that the presence of the retrograde vector and the aerosolized spray do not influence baseline coughs compared with naïve animals. In a second session, all cohorts are then given 1 mg/kg CNO 30 minutes prior to being placed in the acid gas chamber. The number, frequency and intensity of coughs are then quantified and compared both between groups and within groups under both vehicle and CNO conditions. This confirms that guinea pigs which received the AAV with the inhibitory DREADD had reduced coughs when exposed to the acid gas following administration of the CNO regulator compared with prior to administration of CNO and compared with the mCherry and naïve control groups.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method of preventing, inhibiting or treating a cough in a mammal, comprising: providing a mammal having a parasympathetic nerve fiber that innervates the lung which is infected with a viral vector expressing a gene encoding a protein, the activity of which is inhibited by an agent or energy; and administering the agent or delivering the energy to the mammal in an amount effective to inhibit the activity of the protein, thereby preventing, inhibiting or treating cough in the mammal.

2. The method of claim 1, wherein the viral vector is administered to the mammal via inhalation or injection.

3. The method of claim 1, wherein the agent is intravenously administered.

4. The method of claim 1, wherein the mammal is a human.

5. The method of claim 1, wherein the mammal has idiopathic cough, intractable cough, chronic obstructive pulmonary disease (COPD) or gastric reflux.

6-8. (canceled)

9. The method of claim 1, wherein the viral vector is a retrograde form of adeno-associated virus, lentivirus or canine adenovirus or is modified for retrograde transport.

10. The method of claim 1, wherein the gene encodes a light-sensitive ion channel (optogenetics), a chemically-responsive ion channel (chemogenetics), an ultrasound-sensitive ion channel (sonogenetics), a magnetic-field responsive ion channel (magnetogenetics) or a designer receptor exclusively activated by designer drugs (DREADDs).

11-13. (canceled)

14. A method of delivering genes to nerve fibers controlling visceral organ function in a mammal, comprising: administering an agent or delivering energy to a mammal, the regulatory nerve of which innervates the visceral organ and is infected with a viral vector comprising a gene encoding a gene product, the activity of which protein is inhibited or activated by administration of the agent or the delivery of the energy, wherein the amount of the agent administered or the energy delivered is effective to control the visceral organ function.

15. The method of claim 14, wherein the nerve fiber is the vagus nerve, cardiopulmonary nerve, thoracic splanchnic nerve, lumbar splanchnic nerve, sacral splanchnic nerve or pelvic splanchnic nerve.

16. The method of claim 14, wherein the mammalian organ is a stomach, duodenum, intestine, pancreas, liver, lung, heart, adrenal, kidney, gonad, or bladder.

17. (canceled)

18. The method of claim 14, wherein the viral vector provides for retrograde transport in neurons or is modified to provide for retrograde transport in the central nervous system.

19-20. (canceled)

21. The method of claim 14, wherein the gene product regulates the activity of the nerve controlling the organ in response to the administration of the agent.

22. The method of claim 14, wherein the gene encodes a light-sensitive ion channel, a chemically-responsive ion channel, an ultrasound-sensitive ion channel, a magnetic-field responsive ion channel, a designer receptor exclusively activated by designer drugs (DREADDs), channel rhodopsin, nicotinic acetylcholine receptor, gramicidin A, a voltage-gated potassium channel, an ionotropic glutamate receptor, alpha-hemolysin, or a mechanosensitive channel, wherein the gene product blocks toxic protein spread to the vagus nerve of the mammal or wherein the gene product comprises or encodes small hairpin RNA (shRNA), microRNA (miRNA), CrispR/Cas9, an antibody, a single-chain antibody, or an intrabody.

23. (canceled)

24. The method of claim 14 wherein the viral vector is delivered to the vagus nerve.

25. The method of claim 14 wherein the agent administered or the energy delivered allows for control of food intake, control of anal sphincter or control of urinary sphincter in the mammal.

26-27. (canceled)

28. The method of claim 25 wherein the viral vector is taken up retrograde from the gastrointestinal system.

29. The method of claim 22, wherein the toxic protein comprises alpha-synuclein, tau, beta-amyloid, or huntingtin.

30. (canceled)

31. The method of claim 14, wherein the amount administered or delivered prevents or inhibits spread of a toxic protein from the gastrointestinal tract to the brain in a mammal.

32-34. (canceled)

35. The method of claim 14 wherein the viral vector is a rAAV comprising an adeno-associated viral capsid comprising two or more different AAV capsid serotypes.

36. (canceled)

37. A recombinant AAV (rAAV) comprising a capsid formed of capsid proteins from two or more different AAV capsid serotypes.

38-45. (canceled)
